SEQUENCE LISTING (39 sequences)

SEQ ID NO: 1 - DNA, 1286 bp, Saccharomyces cerevisiae
gcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggatccgagcttggaattcgacaggttatcagcaacaacacagtcatatccattctcaattagctctaccacagtgtgtgaaccaatgtatccagcaccacctgtaaccaaaacaattttagaagtactttcactttgtaactgagctgtcatttatattgaattttcaaaaattcttactttttttttggatggacgcaaagaagtttaataatcatattacatggcattaccaccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaactataatgactaaatctcattcagaagaagtgattgtacctgagttcaattctagcgcaaaggaattaccaagaccattggccgaaaagtgcggaattccaagcttggccaagcccggatccggagcttggctgttgcccgtctcactggtgaaaagaaaaaccaccctggcgcccaatacgcaaaccgcctctccccgcgcgttggccgattcattaatgcagctggcacgacaggtttcccgacttaatcgccttgcagcacatccccctttcgccagctggcgtaatagcgaagaggc

SEQ ID NO: 2 - DNA, 31 bp, Artificial
  Primer
acgggtaccgcaaagggaagggatgctaagg

SEQ ID NO: 3 - DNA, 30 bp, Artificial
  Primer
atcggtacctgaacgttacagaaaagcagg

SEQ ID NO: 4 - DNA, 31 bp, Artificial
  Primer
actactagtgcctcttcgctattacgccagc

SEQ ID NO: 5 - DNA, 27 bp, Artificial
  Primer
agaactagtggaagatcgcactccagc

SEQ ID NO: 6 - DNA, 31 bp, Artificial
  Primer
acaactagtaacttttcggccaatggtcttg

SEQ ID NO: 7 - DNA, 37 bp, Artificial
  Primer
actactagtcctatagttttttctccttgacgttaaa

SEQ ID NO: 8 - DNA, 681 bp, Saccharomyces cerevisiae
atgagtagatcttctaagaacgtcatcaaggaattcatgagattcaaggttaaaatggaaggtactgttaacggccacgaattcgaaatcgaaggtgaaggtgagggtagaccatatgaaggtcacaacacagtcaagttgaaggttactaagggtggtccactgccattcgcttgggacatcttgtctccacaattccaatacggttctaaggtctacgtcaagcacccagctgacattccagactacaagaagttgtccttcccagaaggtttcaagtgggaaaggatcatgaacttcgaagacggtggcgttgttactgttactcaagactcctccttgcaagacggttgtttcatctacaaggtcaagctcattggtgtcaacttcccatctgacggtccagtcatgcaaaagaagactatgggttgggaagcttctaccgaacgtttgtacccaagagacggtgtcttgaagggtgaaatccacaaggccttgaagttgaaggacggtggtcactacttggtcgaattcaagtctatctacaaggccaagaagcaagtccaattgccaggctattactacgttgactctaagttggacatcatctctcacaacgaagactacactatcgtcgaacaatacgaacgtactgaaggtagacaccacttgttcttgtaa

SEQ ID NO: 9 - DNA, 34 bp, Artificial
  Primer
actactagttatgagtagatcttctaagaacgtc

SEQ ID NO: 10 - DNA, 33 bp, Artificial
  Primer
tattccgcggttacaagaacaagtggtgtctac

SEQ ID NO: 11 - DNA, 781 bp, Artificial
  Codonoptimierte DNA fr cobA mit Resten aus der Klonierungsite
tcatgaccactactttattgccaggtactgttactttggttggtgcaggtccaggtgatccagaattggttacagttgctggtttaagagcagttcaacaagctgaagttattctatacgatagattggctccacaagatttgttgtctgaagcatctgacgatgcagaattagttccagttggtaaaattccaagaggacattacgttccacaggaggaaattaatcaattattggttgctcatgctagagaaggtagaaaagtagttagattaaagggtggagattctttcgtttttggtagaggtggagaggaatggcaagcatgtgctgaagctggtattccagttagagttattccaggagtttcttctgctactgctggtccagctttggctggtattcctttgactcatagacatttggttcaaggttttactgttgtttcaggtcatgtttctccatcagatgaaagatcagaagttccttggagacaattggctaaggatagattgactttggttattttaatgggagttgctcatatgagagatattgctccagaattgatggctggaggtttaccagcagatactcctgttagagtagtttctaatgcttctttggcttctcaggaatcttggagaactactttgggagatgcagttgctgatatggatgctcatcacgttagaccacctgctttggtagtagttggtactttggctggagtagatttgtctcatccagatcatagagcaccatcagatcataactcgag

SEQ ID NO: 12 - DNA, 1782 bp, Saccharomyces cerevisiae
atggtacgagacttagtgacattgccttcatcactgcccttgattactgctggttttgctactgatcaggttcatttgcttattggtacagggtccacggactcagtaagcgtttgtaagaatagaatccactccattttgaatgctggtggtaatcccatagtagtgaatccctcgtcaccaagccatactaaacaattacaattggaatttggtaagtttgcaaagttcgaaatagtagaaagggagtttaggttatctgatttaactactttggggagagttctggtatgcaaggtagtggatagagtattcgtagatctacccataacacaaagtcgcctatgcgaggagatcttttggcaatgccaaaaactgagaattcccataaatacattccacaaaccagagttttctaccttcaatatgattcctacgtgggtcgacccaaaaggaagtggtttacaaatctcagttactacgaatgggaatggatacatcttggcaaacaggataaaaagagatataatatcacacttacctccaaacatatctgaggtggtgataaacatggggtatttgaaagaccgtattataaacgaagaccataaggccttgttagaggaaaagtactaccagactgacatgtcattacctggatttggctacggcttagatgaggacggttgggagagccataagtttaataagctaattcgtgaatttgaaatgaccagtagagaacagagacttaagagaaccagatggttatctcagataatggagtattacccgatgaacaagctgagtgacatcaagttggaagatttcgagacttcatcttctccaaataaaaagacaaagcaggaaactgtcacagagggtgtagtacctcctaccgatgaaaatattgaaaacggtacaaaacaactacaattatcggaagtgaaaaaagaggagggacctaaaaaactagggaagatttctttagtcggaagtggtccaggctcggtatctatgctaacgataggtgcattacaagaaataaagtctgcagatataatactggcagataaactggtaccgcaagccattttagatttaatacctccaaaaactgaaaccttcatagccaaaaaatttcccggtaatgcagaacgagcacaacaggaattactagctaaaggtttagaatcgttggataatggattgaaagtagtccgtttgaagcaaggtgatccgtatatttttggccgtggtggcgaggaatttaatttcttcaaagatcacggatatattcctgtggttttaccgggcataagctcatccctagcttgtactgtattggctcagatacccgctactcaacgtgatatagcagaccaagtgctcatatgtactgggactgggagaaagggcgctctgcctataattcctgaatttgttgaaagcagaaccaccgtctttctaatggcactgcatcgcgccaacgttctgatcacgggattattgaagcatggctgggatggtgatgtccccgctgcaattgtcgagagaggatcgtgccctgaccagcgtgttactagaactcttcttaaatgggtaccagaagtcgtggaggagattggttcaaggccccccggtgtcttggttgtaggcaaggctgtgaatgcattggttgaaaaagatctgataaattttgacgaatcaagaaaatttgtcattgatgaaggttttagagaatttgaggttgatgtagatagtctatttaagttatactaa

SEQ ID NO: 13 - DNA, 32 bp, Artificial
  Primer
aattatccatggtacgagacttagtgacattg

SEQ ID NO: 14 - DNA, 45 bp, Artificial
  Primer
aattaactcgagttgtataacttaaatagactatctacatcaacc

SEQ ID NO: 15 - DNA, 36 bp, Saccharomyces cerevisiae
atccttgagctctcagcagctctgatgtagatacac

SEQ ID NO: 16 - DNA, 43 bp, Saccharomyces cerevisiae
atcccccatggctgataatgggttagtagtttataattatgtg

SEQ ID NO: 17 - DNA, 488 bp, Saccharomyces cerevisiae
tcagcagctctgatgtagatacacgtatctcgacatgttttatttttactatacatacataaaagaaataaaaaatgataacgtgtatattattattcatataatcaatgagggtcattttctgaaacgcaaaaaacggtaaatggaaaaaaaataaagatagaaaaagaaaacaaacaaaggaaaggttagcatattaaataactgagctgatacttcaacagcatcgctgaagagaacagtattgaaaccgaaacattttctaaaggcaaacaaggtactccatatttgctggacgtgttctttctctcgtttcatatgcataattctgtcataagcctgttctttttcctggcttaaacatcccgttttgtaaaagagaaatctattccacatatttcattcattcggctaccatactaaggataaactaatcccgttgttttttggcctcgtcacataattataaactactaacccattatcag

SEQ ID NO: 18 - DNA, 33 bp, Saccharomyces cerevisiae
atccccgcggtagcttcaaaatgtttctactcc

SEQ ID NO: 19 - DNA, 38 bp, Saccharomyces cerevisiae
atcccccatggtttgtaattaaaacttagattagattg

SEQ ID NO: 20 - DNA, 411 bp, Saccharomyces cerevisiae
tagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 21 - DNA, 36 bp, Saccharomyces cerevisiae
atccccgcggcagttcgagtttatcattatcaatac

SEQ ID NO: 22 - DNA, 36 bp, Saccharomyces cerevisiae
atcccccatggtttgtttgtttatgtgtgtttattc

SEQ ID NO: 23 - DNA, 680 bp, Saccharomyces cerevisiae
cagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 24 - DNA, 45 bp, Artificial
  Primer
ctatataactctgtagaaataaagagtatcatctttcaaagagct

SEQ ID NO: 25 - DNA, 45 bp, Artificial
  Primer
ctttgaaagatgatactctttatttctacagagttatatagacgt

SEQ ID NO: 26 - DNA, 32 bp, Artificial
  Primer
aattccacatgtccgacccgagtaagacaagc

SEQ ID NO: 27 - DNA, 40 bp, Artificial
  Primer
attgccgcggttagtcgacatctagaaaatctacttgaag

SEQ ID NO: 28 - DNA, 50 bp, Artificial
  Primer
tgaatactcatactcttcctgtttaaacattattgaagcatttatcaggg

SEQ ID NO: 29 - DNA, 52 bp, Artificial
  Primer
ttaaatcaatctaaagtatatatgtttaaacttggtctgacagttaccaatg

SEQ ID NO: 30 - DNA, 47 bp, Artificial
  Primer
aaaaaaccgtttaaacaggaagagtatgattgaacaagatggattgc

SEQ ID NO: 31 - DNA, 50 bp, Artificial
  Primer
aaaaaaccgtttaaacttggtctgacagtcagaagaactcgtcaagaagg

SEQ ID NO: 32 - DNA, 41 bp, Artificial
  Primer
ctgccttatgaaannkctcaaggtgaggggcctgcaaccag

SEQ ID NO: 33 - DNA, 41 bp, Artificial
  Primer
ccctcaccttgagmnntttcataaggcagtcatgcaagctc

SEQ ID NO: 34 - DNA, 678 bp, Discosoma sp.
atgagtagatcttctaagaacgtcatcaaggaattcatgagattcaaggttaaaatggaaggtactgttaacggccacgaattcgaaatcgaaggtgaaggtgagggtagaccatatgaaggtcacaacacagtcaagttgaaggttactaagggtggtccactgccattcgcttgggacatcttgtctccacaattccaatacggttctaaggtctacgtcaagcacccagctgacattccagactacaagaagttgtccttcccagaaggtttcaagtgggaaaggatcatgaacttcgaagacggtggcgttgttactgttactcaagactcctccttgcaagacggttgtttcatctacaaggtcaagctcattggtgtcaacttcccatctgacggtccagtcatgcaaaagaagactatgggttgggaagcttctaccgaacgtttgtacccaagagacggtgtcttgaagggtgaaatccacaaggccttgaagttgaaggacggtggtcactacttggtcgaattcaagtctatctacaaggccaagaagcaagtccaattgccaggctattactacgttgactctaagttggacatcatctctcacaacgaagactacactatcgtcgaacaatacgaacgtactgaaggtagacaccacttgttcttg

SEQ ID NO: 35 - DNA, 771 bp, Saccharomyces cerevisiae
atgaccactactttattgccaggtactgttactttggttggtgcaggtccaggtgatccagaattggttacagttgctggtttaagagcagttcaacaagctgaagttattctatacgatagattggctccacaagatttgttgtctgaagcatctgacgatgcagaattagttccagttggtaaaattccaagaggacattacgttccacaggaggaaattaatcaattattggttgctcatgctagagaaggtagaaaagtagttagattaaagggtggagattctttcgtttttggtagaggtggagaggaatggcaagcatgtgctgaagctggtattccagttagagttattccaggagtttcttctgctactgctggtccagctttggctggtattcctttgactcatagacatttggttcaaggttttactgttgtttcaggtcatgtttctccatcagatgaaagatcagaagttccttggagacaattggctaaggatagattgactttggttattttaatgggagttgctcatatgagagatattgctccagaattgatggctggaggtttaccagcagatactcctgttagagtagtttctaatgcttctttggcttctcaggaatcttggagaactactttgggagatgcagttgctgatatggatgctcatcacgttagaccacctgctttggtagtagttggtactttggctggagtagatttgtctcatccagatcatagagcaccatcagatcat

SEQ ID NO: 36 - DNA, 1779 bp, Saccharomyces cerevisiae
atggtacgagacttagtgacattgccttcatcactgcccttgattactgctggttttgctactgatcaggttcatttgcttattggtacagggtccacggactcagtaagcgtttgtaagaatagaatccactccattttgaatgctggtggtaatcccatagtagtgaatccctcgtcaccaagccatactaaacaattacaattggaatttggtaagtttgcaaagttcgaaatagtagaaagggagtttaggttatctgatttaactactttggggagagttctggtatgcaaggtagtggatagagtattcgtagatctacccataacacaaagtcgcctatgcgaggagatcttttggcaatgccaaaaactgagaattcccataaatacattccacaaaccagagttttctaccttcaatatgattcctacgtgggtcgacccaaaaggaagtggtttacaaatctcagttactacgaatgggaatggatacatcttggcaaacaggataaaaagagatataatatcacacttacctccaaacatatctgaggtggtgataaacatggggtatttgaaagaccgtattataaacgaagaccataaggccttgttagaggaaaagtactaccagactgacatgtcattacctggatttggctacggcttagatgaggacggttgggagagccataagtttaataagctaattcgtgaatttgaaatgaccagtagagaacagagacttaagagaaccagatggttatctcagataatggagtattacccgatgaacaagctgagtgacatcaagttggaagatttcgagacttcatcttctccaaataaaaagacaaagcaggaaactgtcacagagggtgtagtacctcctaccgatgaaaatattgaaaacggtacaaaacaactacaattatcggaagtgaaaaaagaggagggacctaaaaaactagggaagatttctttagtcggaagtggtccaggctcggtatctatgctaacgataggtgcattacaagaaataaagtctgcagatataatactggcagataaactggtaccgcaagccattttagatttaatacctccaaaaactgaaaccttcatagccaaaaaatttcccggtaatgcagaacgagcacaacaggaattactagctaaaggtttagaatcgttggataatggattgaaagtagtccgtttgaagcaaggtgatccgtatatttttggccgtggtggcgaggaatttaatttcttcaaagatcacggatatattcctgtggttttaccgggcataagctcatccctagcttgtactgtattggctcagatacccgctactcaacgtgatatagcagaccaagtgctcatatgtactgggactgggagaaagggcgctctgcctataattcctgaatttgttgaaagcagaaccaccgtctttctaatggcactgcatcgcgccaacgttctgatcacgggattattgaagcatggctgggatggtgatgtccccgctgcaattgtcgagagaggatcgtgccctgaccagcgtgttactagaactcttcttaaatgggtaccagaagtcgtggaggagattggttcaaggccccccggtgtcttggttgtaggcaaggctgtgaatgcattggttgaaaaagatctgataaattttgacgaatcaagaaaatttgtcattgatgaaggttttagagaatttgaggttgatgtagatagtctatttaagttatac

SEQ ID NO: 37 - DNA, 771 bp, Saccharomyces cerevisiae
atggaaacgaccgaacagttaatcaacgaaccgctcgaccatcgcggtgaagtggtgctggttggtgcaggtccgggcgatgccgggctgctgacactgaaaggactgcaacaaattcagcaggcagatgtggtggtctacgaccgtctggtttctgacgatattatgaatctggtacgccgcgatgcggaccgtgttttcgtcggcaaacgcgcgggataccactgcgtaccccaggaagagattaaccagatcctgctgcgggaagcgcaaaaaggcaaacgcgtggtgcggctgaaaggtggcgatccgtttatttttggccgtggtggcgaagagctggaaacactgtgcaacgcgggtattccgttctcggtggttccgggtattaccgcagcttctggttgctctgcctattcgggtattccactcacgcatcgcgattatgcccagagcgtacgcttaattaccggacatttaaaaaccggtggcgagctggactgggaaaacctggcggcagaaaaacagacgctggtgttctatatggggttgaatcaggccgcgactattcagcaaaagctgattgaacacggaatgccaggcgaaatgccggtggcaattgtcgaaaacggtacggcagtcacgcagcgcgtgattgacggtacgctcacacagctgggagaactggcgcagcaaatgaacagtccatcgctaattattattggtcgggttgttggcctgcgcgataaactgaactggttctccaaccat

SEQ ID NO: 38 - DNA, 32 bp, Artificial
  Primer
ccaaccccatggaaacgaccgaacagttaatc

SEQ ID NO: 39 - DNA, 35 bp, Artificial
  Primer
aatgttctcgagttatggttggagaaccagttcag

(19) United States
(12) Patent Application Publication
(10) Pub. No.: US 2009/0163370 A1
(43) Pub. Date: Jun. 25, 2009

(54) Competitive N-Hybrid System

(75) Inventors: Claudia Arntz, Lengerich (DE); Danielle Meinders, Steinfurt (DE); Christoph Block, Münster (DE)

Correspondence Address: HENRY M FEIEREISEN, LLC;HENRY M FEIEREISEN, 708 THIRD AVENUE, SUITE 1501, NEW YORK, NY, 10017, US

(73) Assignee: Signalomics gmbH, Wien (AT)

(21) Appl. No.: 11/913,647

(22) PCT Filed: May 4, 2006
(86) PCT No.: PCT/EP2006/004190

§ 371 Date: Jun. 27, 2008

(30) Foreign Application Priority Data
May 4, 2005 (EP) 05009771.6

(51) Int. Cl. C40B 30/04; C40B 40/02; C12N 15/00

(52) U.S. Cl. 506/9; 506/14; 435/320.1

(57) ABSTRACT

The invention relates to a method for identifying highly affinous ligands comprising the following steps: a) creating a bank for a mutagenized first hybrid protein comprising a plurality of mutants; b) expression of a first hybrid protein in a host with a second hybrid protein, one of the hybrid proteins comprising the DNA binding domain of a transcription factor and a bait protein and the other hybrid protein comprising the activation domain for a transcription factor and a prey protein; c) allowing the binding reaction between the first and second hybrid protein in order to form a complex with a functional transcription factor in the host cell under reaction conditions, which are such that the balance of the binding reaction is offset on the side of the hybrid proteins; d) detecting the binding reaction by detecting a reporter gene expressed via the functional transcription factor; e) optionally repeating one or more steps from a) to d); f) selection of a mutant.

The invention relates to a development of the two-hybrid system for identifying high-affinity ligand interactions and claims the priority of the European patent application 05 009 771.6 whose contents reference is made to.

The two-hybrid system was originally developed by Fields and Song in 1989 and became very popular and was used extensively owing to the possibility of identifying with the aid thereof an interaction partner for a particular protein from a gene library by screening in S. cerevisiae. Its essential features are based on fusing the transactivating activity of a transcription factor whose polypeptide chain has initially been split into a separate DNA binding domain and a transactivating domain in each case to a protein (bait protein and prey protein, respectively) and functionally reconstituting said activity by way of subsequent noncovalent interaction of said fusion proteins (hybrid proteins).

Since the original conception of the two-hybrid system (Yeast Two Hybrid) was unable to take into account posttranslational modifications of the interacting proteins, said system was extended to the “three-hybrid system” a few years later. This made it possible, for example by expressing a third component such as a protein kinase for example, to detect protein-protein interaction as a function of phosphorylation by a protein kinase (Osborne et al., 1995). It was likewise demonstrated that a three-hybrid system can detect the presence of a partner essential to said protein-protein interaction, which partner is involved in the formation of a ternary complex (Zhang and Lautar, 1996).

In their original configuration, the two- and three-hybrid systems involved expressing a gene essential to growth of the yeasts by reconstituting the transcription factor by way of successful interaction of the fusion proteins (forward hybrid system). Later, binding was detected by employing a reporter gene (e.g. lacZ) which enabled reporter gene-expressing colonies to be readily identified or else—with using a soluble substrate—lacZ activity to be quantified by way of a color reaction of a substrate (e.g. X-Gal) with precipitating product (BioTechniques 2000, 29, 278-288, Jaitner et al., 1997).

Another development was a reversal of the original screening approach (reverse n-hybrid), in which a toxic gene is transcribed due to a protein-protein interaction and only disruption of said protein-protein interaction enables the yeast cells to grow (Vidal et al., 1996). However, inhibition of the interaction was also achieved in the forward n-hybrid by expressing an inhibitor (Tirode et al., 1997).

However, the use of the hybrid system is not limited to three components. Probably any protein complexes having sufficient interaction affinity are suitable for the hybrid system in principle. Thus the use of a four-hybrid system has already been demonstrated (Sandrock and Egly, 2001). Four components in a single system are also used in the “dual bait” two-hybrid system. Here, in each case two interacting two-hybrid pairs with different reporter genes are employed, in order to enable specific and unspecific interactions in two-hybrid screening to be discriminated more quickly (Serebriiskii et al., JBC 1999). This approach simplifies qualitative determination of the specificity of interaction partners found in the two-hybrid system.

In the classical two-hybrid system, protein-protein interaction takes place inside the nucleus. The latter, however, is not a suitable cell compartment for all protein-protein interactions. For interactions for which the nucleus is an unsuitable location, systems were developed in which the interactions to be detected take place, for example, either in the cytoplasma or at the cell membrane (Aronheim et al., 1997).

The simultaneous use of a selective growth marker (e.g. His3) and an enzymic reporter gene (e.g. lacZ) for the established color and fluorescent substrates was an early attempt at employing the two-hybrid system not only for detecting protein-protein interactions—i.e. as a qualitative approach—, but also at utilizing it for quantifying interactions. However, a first comparative study using various transcription factors was able only to demonstrate suitability of the two-hybrid system for semiquantitative studies (Estojak et al., 1995). A study using point mutants revealed a quantitative correlation in the two-hybrid system between in vivo and in vitro data in the nano- to micromolar affinity range (Jaitner et al., 1997).

The problem addressed in these studies of quantitative determination of protein-protein interactions in the two-hybrid system was taken up again in a more recent study which investigated the very broad usage of n-hybrid systems in interaction screening in the field of medicament development (de Felipe et al., 2004).

In the field of medicament development and the development of diagnostics it is particularly important to be able to quantify the possible interactions between the binding partners involved over a very wide affinity range (“broad dynamic range”) and to detect at the same time the specificity of said binding interactions, since pharmaceutical active compounds or pharmaceutically utilizable proteins should have very high affinity and very high specificity.

The current work of Felipe et al. (2004) however, clearly shows the limits of the known hybrid systems in this respect. In fact, the dynamic range available for said affinity studies merely extends over one to two orders of magnitude. In order to be able to cover the entire range required, a multiplicity of different systems would therefore have to be used.

Some studies confirm these statements. Said studies are based on investigations regarding the correlation between the biochemical affinity of a prey protein to the bait protein and the quantitative result of interaction analysis via the detected amount of reporter gene expressed (Readout) in the two-hybrid system. The interaction investigated is the binding between Ras and the Ras-binding domain (RafRBD) of the Raf protein kinase. While the two-hybrid system was able to purely distinguish RafRBD mutants having diminished affinity for Ras from RafRBD wild type (RafRBD-wt) (Jaitner et al., 1997), all attempts at identifying an RafRBD mutant described in the literature as having increased affinity for Ras (RafRBD-A85K, Burgess et al., 2000)—as was confirmed also in our own biochemical measurements—by way of increased reporter gene activity in the two-hybrid system failed. It has therefore not been possible up to now to distinguish the use of the RafRBD mutant, RafRBD-A85K, having increased affinity for Ras according to biochemical measurements, from the wild-type form, RafRBD-wt, in the two-hybrid system. This suggests that said mutant is outside the dynamic range of the two-hybrid system.

In order to bb able nevertheless to identify a high-affinity binding partner, it would be conceivable to establish various different n-hybrid systems to thereby achieve, in the overall view, a screening over the desired wide dynamic range, and in particular also to allow proteins having therapeutically and diagnostically relevant high affinities to be identified. This can hardly be done in practice, however. More specifically, this cannot guarantee that proteins having correspondingly improved properties can be identified reliably in the screenings, since the limits of the dynamic range cannot be predicted in detail.

It is therefore the object of the present invention to further develop the known hybrid system in that a greater dynamic range is available for the affinity studies, particularly in order to be able to select thereby high-affinity ligand interactions.

This object is achieved by a method as claimed in claim 1. Advantageous developments are in each case a subject matter of the dependent claims and of the independent subclaims.

The invention is based on the idea of carrying out the known hybrid system in such a way that the binding reaction between the first ligand (first hybrid protein or fusion protein) and the second ligand (second hybrid protein or fusion protein) is deliberately “made worse” by choosing suitable reaction conditions. A worsening in accordance with the present invention takes place whenever the equilibrium (dynamic equilibrium) of the reaction of the formation of a ligand complex shifts in favor of the ligands (reactants). The binding reaction between the ligands is therefore inhibited or slowed down. The ligand complex is defined with respect to the ligands by a functional transcription factor.

If, however, either of the ligands of the starting system is replaced, for example, with a ligand having a distinctly higher affinity for the in each case other binding partner, the “worsening” of said system is overcome, with correspondingly more ligand complexes being formed compared to the starting situation. The use of a high-affinity ligand therefore results, compared to the disrupted starting situation, in increased expression of the reporter gene whose activity can be detected quantitatively. This enables in particular the relative affinity of an interaction pair to be depicted in comparison with a comparative pair.

By modifying the reaction conditions, it is possible to adapt or increase the dynamic range within which the affinity studies are possible, depending on the problem of interest and the desired affinity of the sought-after ligand. Thus it is possible for a starting library of fusion proteins to be subjected to a plurality of cycles of the method of the invention, with each cycle being repeatable under altered conditions. Said library of fusion proteins is generated by random or directed mutagenesis beforehand. The method of the invention therefore provides a system which selects, for example, proteins with high affinity for the bait protein. In this repetitive usage, the method of the invention can thus serve as a selection method and provide ligands/mutants having a theoretically unlimited high affinity.

The method of the invention is suitable especially as a screening method for comparing affinity and high-affinity ligands. In order to enable said comparison, reporter gene expression of a wild type ligand may be set as a reference value (100%), for example. From this starting point, the affinities of mutants of said wild type can be depicted as parameters relative to the affinity of said wild type.

The ligand binding equilibrium can be influenced in many ways and thus also be impaired deliberately. Thus the ionic strength of the reaction medium may be varied in order to generate in particular mutants whose association kinetics have been modified by the number of ionic amino acids and complementary surface charges. Alteration of the pH can influence both association kinetics and dissociation kinetics for at least one of the ligands. As a result, mutants with optionally protonatable or non-protonatable amino acid side chains are selected that achieve high affinity under particular physiology pH conditions.

In a particularly advantageous embodiment of the invention, a competitor is used for “worsening” the binding reaction. Said competitor binds to one of the hybrid proteins and thereby inhibits or delays in the manner of a competitive or non-competitive inhibitor formation of the ligand complex. According to the invention it is possible to use a competitor both to the prey protein and to the bait protein. If a competitor to the prey protein were to be chosen—and thereby the formation of the ligand complex basically to be impaired—and the affinity of said prey protein for the bait protein should exceeds the affinity of the competitor used for said bait protein, the equilibrium of the ligand binding reaction will be influenced in favor of ligand complex binding. As a result, the high-affinity prey protein can be detected quantitatively by way of correspondingly high reporter gene expression. This high-affinity reaction would not be detectable if the detection limit of the system had already been exceeded.

In this case, fine adjustment of the system can be influenced decisively by the choice of the competitor and its affinity for the hybrid protein. The concentration of said competitor is also of considerable importance, since the concentration of a reactant is known to determine the equilibrium of a reaction to a considerable extent.

A particular advantage of this embodiment is the fact that the choice of a competitor which is specific per se is also associated with an increase in specificity of the ligand identified by said repetitive selecting (e.g. prey protein). In fact, by using a protein similar to the native binding partner of the bait protein—which protein accordingly has a correspondingly high specificity—, any prey proteins having a low specificity will be left out of consideration subsequently. Consequently, prey proteins having “unspecific binding” are excluded.

In a further, particularly preferred development of the method of the invention, the competitor is expressed in the host cell itself. Firstly, this has the advantage of the competitor already being present in the cell and in addition opens up the possibility of varying expression of the competitor by choosing a suitable promoter—and thereby, as a result, varying the concentration of said competitor, which is essential for the position of the equilibrium of the binding reaction. As a result, influencing the method of the invention can be modified both by choosing the competitor and by regulating its expression.

In a particularly advantageous embodiment, the growth conditions, after expression of the competitor in a host cell, are varied by influencing selection markers as a function of the media composition, in order to specifically promote interactions with modified affinity. Thus, for example, the transformed host cells can be cultured on a selective medium containing aminotriazol as competitive inhibitor for His expression. With the same protein-protein interaction, the reporter gene readout corresponds to the high selection pressure on the His3 gene. By adding different concentrations of aminotriazol, only host cells containing fusion protein with sufficiently high affinity are selected. This is because the affinity of the binding partners must be so great that a sufficient amount of His or the reporter gene is still expressed despite growth on a competitive His-expression inhibitor. The concentration of the selection marker in the medium determines—in interaction with the other factors of the system of the invention (e.g. strength of the promoter directing competitive expression)—the desired affinity of the fusion protein selected via the host cell.

Up to now mainly enzymic detection processes (lacZ gene; β-galactosidase assay) or growth on selective medium (e.g. HIS3 or LEU2) are used as reporters (readout) for the TH system. While the enzymic detection processes require the addition of substrates and in some cases also preparation of cell extracts, growth on selective medium does not enable the binding strength between the interacting proteins to be evaluated quantitatively.

For quantitative screening, for example within the framework of a directed or random mutagenesis, however, preference is given to a reporter whose expression is under control of a regulatory promoter and can be measured directly quantitatively and qualitatively. Reporter genes which provide a fluorescent compound in the host organism fulfill these requirements to a particular extent.

Previously only GFP and its (improved) derivative, EGFP, have been used as reporters in the TH system; quantitative evaluation is difficult here, since the maximum fluorescence is within the green range in which autofluorescence of the yeast cells is likewise very high. Moreover, maximum excitation of GFP is in the near UV range (approx. 395 nm), and consequently DNA damage and stress reactions being triggered by the excitation light in the cells cannot be ruled out.

The invention therefore makes use of reporters whose maximum emission is in the red range. The maximum emission is advantageously between 550 and 700 nm, in particular between 580 and 650 nm. In a preferred embodiment, it is in the range from about 600 to about 620 nm, in particular at about 600 to 610 nm. The reporter genes may, where appropriate, have been codon-optimized beforehand by way of adaptation to expression in yeast. The fluorophores formed by the enzymes encoded by the reporter genes are suitable as readout (signal/reporter) in all systems in which at least two hybrid proteins are coexpressed in yeasts. The reporter genes are advantageously coexpressed in the yeasts and are preferably under control of a regulatable promoter.

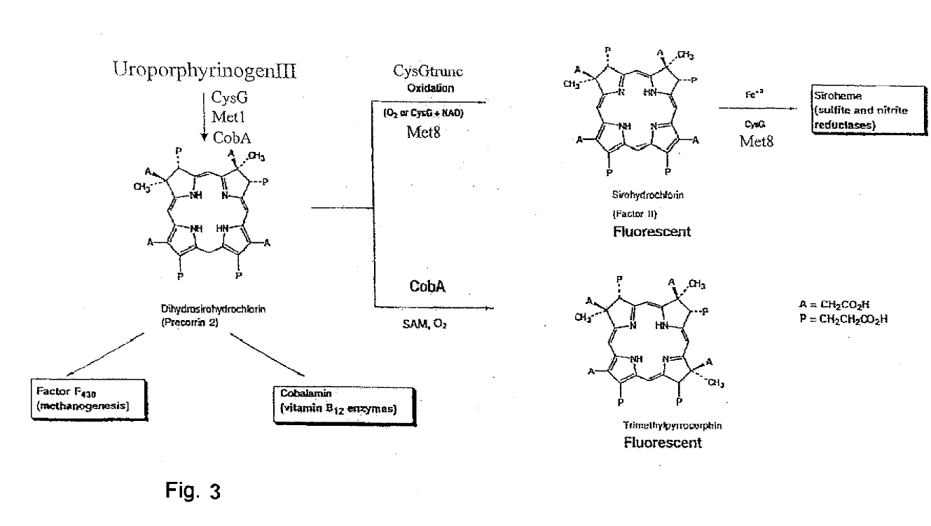

Examples of reporter genes which may be used are CysG and CobA (Roessner, 2002) which in each case yield fluorescent uroporphyrinogen III derivatives (FIG. 3; source organisms of the genes: Propionibacterium freudenreichii: CobA; Saccharomyces cerevisiae: Met1/Met8; Escherichia coli: CysG, according to Roessner 2002).

It is also possible to use fluorescent proteins as readout, for example by way of using phycocyanine (Arntz et al., 2004) or RedStar (Knop et al., 2002).

### EXAMPLES

### 1. Principle of the Method of the Invention with Competitor

In addition to the interacting hybrid proteins (fusion proteins) of the known two-hybrid system, a third component is advantageously expressed in S. cerevisiae. In the case of mutagenesis of the bait protein, said third component is preferably the free wild-type bait protein. In the case of mutagenesis of the prey protein for identifying high-affinity prey proteins, preference is given to expressing the wild-type prey protein as competitor.

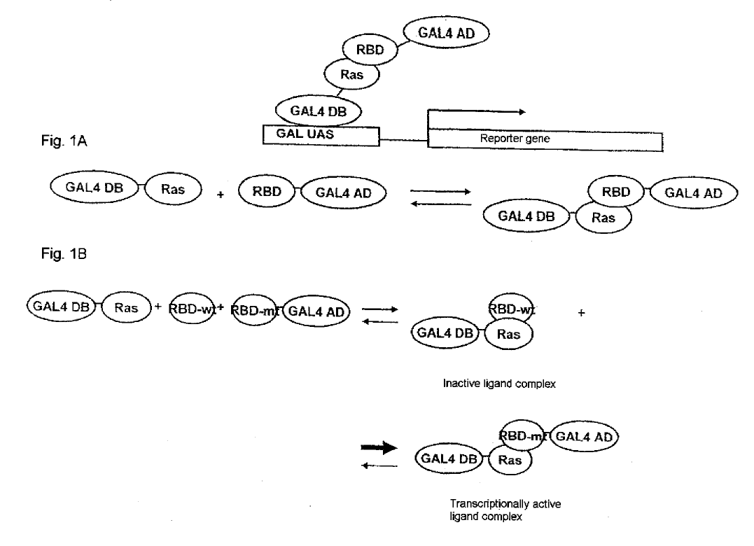

FIG. 1B depicts the basic principle of the method of the invention in this embodiment for identifying affinity RafRBD prey proteins with expression of the wild-type prey protein as competitor. When increasing the affinity of the mutated RafRBD fusion protein (RBD-mt), preference is given to the transcriptionally functional ligand complex being formed over the nonfunctional (inactive) complex of the competitor RafRBD-wt (RBD-wt) with Ras. [Abbreviations. RBD-mt: mutated RafRBD-fusion protein; RafRBD-wt; wild-type-RafRBD; RBD: Ras-binding domain; DB: DNA-binding domain; AD: transactivating domain; UAS: upstream activator sequence].

FIG. 1A depicts the known two-hybrid system. The known system uses only the wild-type variant of the prey protein in the fusion protein. [Abbreviations: RBD: Ras-binding domain; DB: DNA-binding domain; AD: transactivating domain; UAS: upstream activator sequence].

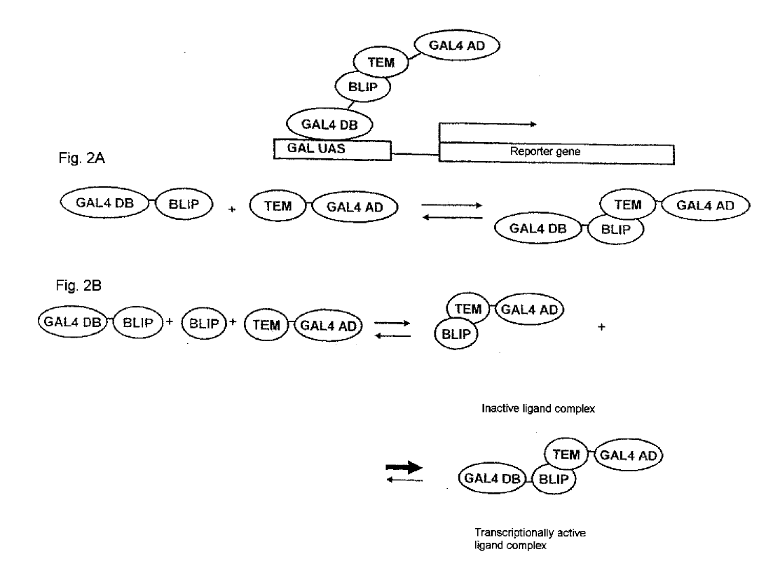

The method according to FIG. 2B serves to investigate the interaction between the proteins BLIP, as bait protein, and TEM, as prey protein (for the prior art, see FIG. 2A). The bait protein has previously been mutagenized (BLIP-mt) and used as fusion protein. This system additionally expresses BLIP wild type (BLIP-wt) which serves as competitor. When increasing the affinity of the mutated BLIP fusion protein, preference is given to forming the transcriptionally functional (active) ligand complex over the inactive ligand complex of BLIP-wt and the fusion protein with TEM. [Abbreviations: DB: DNA-binding domain; AD: transactivating domain; BLIP-wt: bait protein wild type; BLIP-mt: mutated bait protein; UAS: upstream activator sequence].

The results obtained with the aid of the method of the invention using the two-hybrid system of Ras bait protein and RafRDB prey protein, with additional expression of the prey protein wild type as competitor, are illustrated below. The Raf-RBD fusion prey proteins were generated previously by mutagenesis known to the skilled worker. In principle all customary methods are available for mutagenesis.

These results clearly show that the dynamic range of the hybrid system of the invention can be adjusted via the presence of the competitor—RafRBD in the examples illustrated—in each case in such a way that a protein with improved properties can be identified in a reliable manner. Preferred variables for adjusting the dynamic range of this embodiment are the affinity of the competitor and the strength of expression of the competitor (strength of the promoter). It is possible by repeatedly employing this principle in consecutive rounds of selection (repetitive selection), with in each case starting proteins (prey proteins) and competitors having improved affinity and specificity, respectively, to identify theoretically proteins having unlimited high affinity and unlimited high specificity.

The method of the invention therefore involves mutants with improved affinity to be generated which may be used as high-affinity proteins, inter alia for diagnostic or therapeutic purposes. Said mutants may be determined by direct quantitative screening.

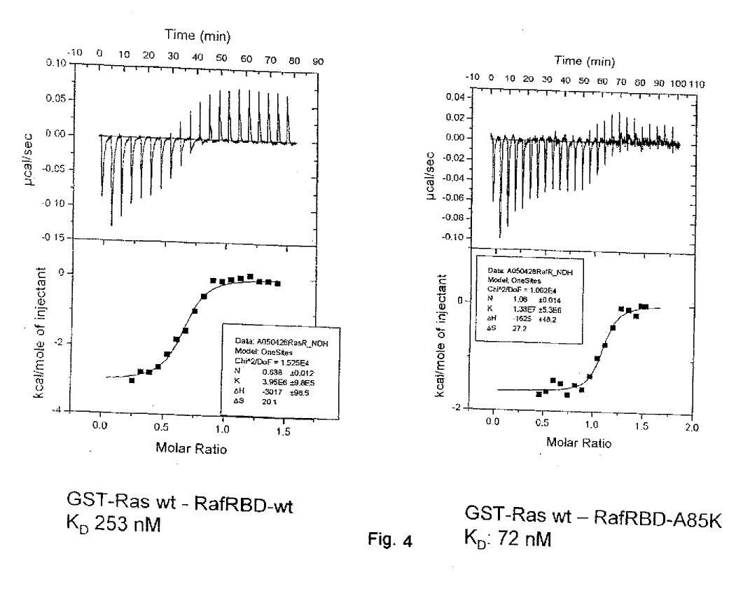

In order to demonstrate the principle of the method of combining the mutagenesis of the prey protein with expression of the prey protein wild type as competitor, the RafRBD mutant, RafRBD-A85K, was used, whose binding affinity has been biochemically characterized previously. According to in-house microcalorimetric measurements, the dissociation constant of the RafRBD-A85K mutant is 72 nM in PBS buffer compared to the dissociation constant of the corresponding RafRBD-wt protein of 253 nM (FIG. 4).

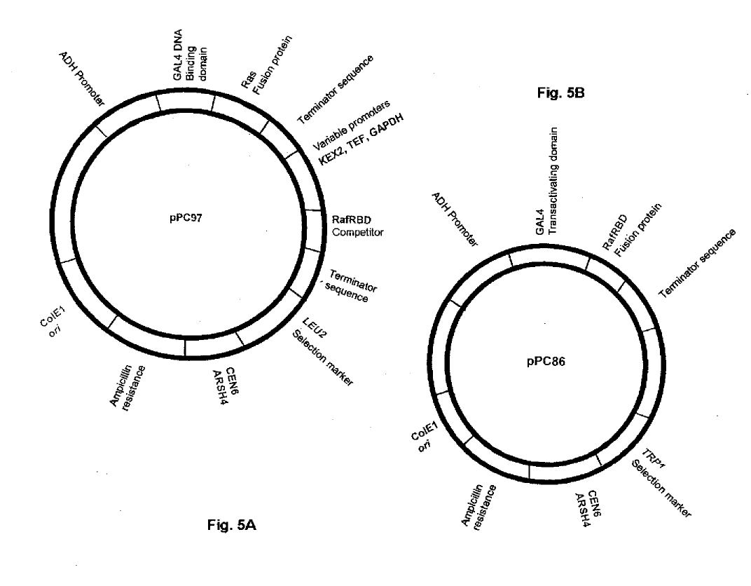

In order to investigate the Ras/Raf interactions in the method of the invention, a plasmid derived from the Ras-Gal4 plasmid may be used for providing the bait fusion protein, pPC97. The competitor is also encoded on this plasmid. FIG. 5A gives an overview over a possible plasmid. FIG. 5B depicts a plasmid based on pPC86 which encodes the prey fusion protein.

### Construction of Yeast Strains for the Method of the Invention

### a) Reporter Gene for Quantitative Screening

The method of the invention requires a reporter gene which is under the control of a regulatable promoter and whose activity can be measured directly, qualitatively and quantitatively in the intact yeast colony. Reporter genes effecting fluorescence as readout meet these requirements.

### (ii) Choosing the Promoter for the Reporter Gene

Expression of the β-galactosidase reporter gene in the yeast Y190 is regulated by the strength of interaction of GAL4 binding and activating domains whose genes are introduced by two different vectors (pPC86 and pPC97, Chevray and Nathans, 1992) into the yeast cell.

The yeast strain Y190 genotype is known as: “Mat a, leu2-3, 112, ura3-52, trp1-901, his3-Δ200, ade2-101, gal4Δgal80A, URA3::GAL-lacZ, LYS2: GAL-HIS3, cyh”. URA3::GAL-lacZ, here means that the promoter in question (GAL; divergent GAL1/GAL10 promoter) has been integrated into the URA3-gene.

The aim of cloning a reporter gene downstream of this promoter and integrating it into the genome of the yeast requires detailed knowledge of the situation at this site in the genome (cf. Yocum et al (1984) on Integration of the YIp plasmid pRy171 into the genome of Y152 (derived from YJ0-Z, Leuther and Johnston, 1992) which is the precursor strain of Y153 from which in turn Y190 is derived).

The generation of plasmid pRy171 which carries the GAL promoter together with the lacZ gene, both downstream of the URA3 gene, was then deciphered in order to obtain sequence data: Yocum et al. (1984) have generated said plasmid from plasmid pLRIΔ3 by removing the sequences of the 2 μm origin of replication. pLRIΔ3 corresponds to plasmid pRy131 apart from an XhoI linker in the middle of the divergent promoter. pRy131 was generated by West et al. (1984) from pLG 669 (Guarente and Ptashne, 1981) and pRy116. pLG 669 in turn is derived from YEp24, a plasmid with pBR322 backbone (Botstein et al., 1979).

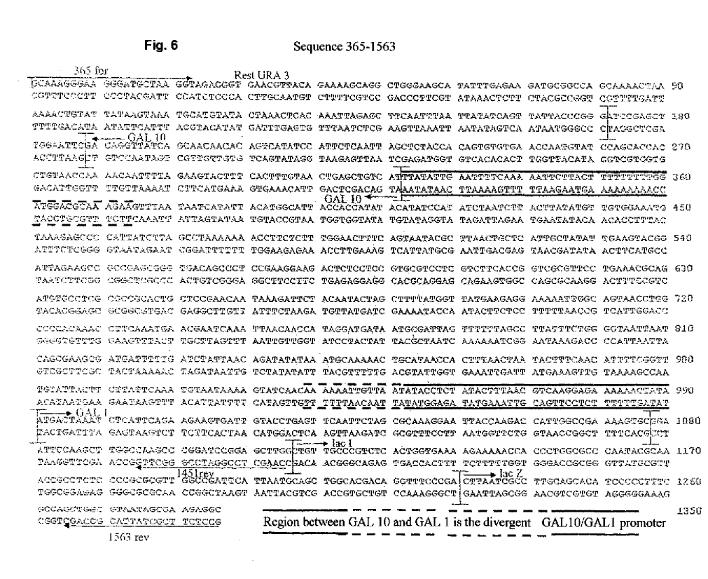

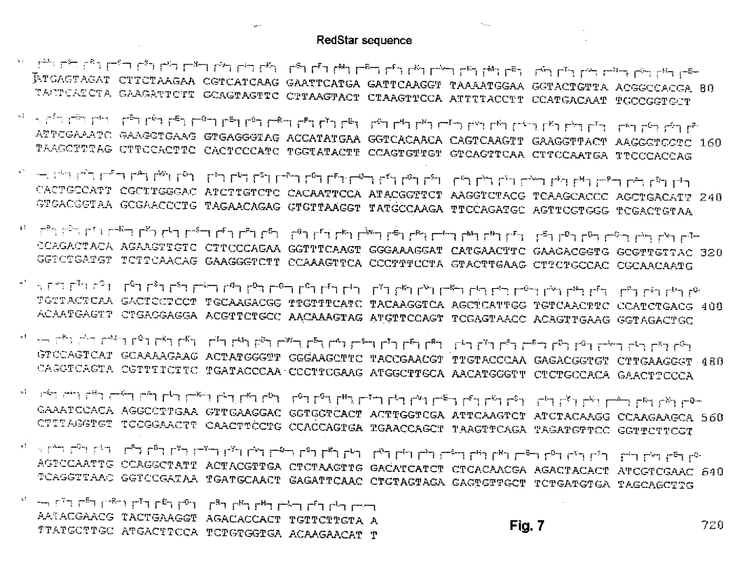

From these data a sequence was generated, according to which primers were synthesized (365-for, 394 for and 1563-rev also and 1674-rev). These were used in a PCR with genomic DNA from Y190 for amplification and sequencing of said piece of DNA. By this the actual sequence of the divergent GAL10/GAL-1 promoter was identified, downstream of which the reporter gene was to be cloned. The sequence of PCR fragment 365-1563 is enclosed (SEQ ID NO 1, FIG. 6, the essential features of the sequence are indicated).

The corresponding pieces of the promoter were amplified by PCR using said primers and then cloned as a fusion product with a fluorophore. In addition, the promoter 365-1451 which no longer has a lacI/5′lacZ portion was also selected. This was done on consideration that additional gene portions might impede expression of the selected fluorophore. Another promoter in which also the GAL1 portion had been reduced to zero (365-1366) was likewise tested. The fluorophore was RedStar (Knop et al., 2002; see section (iii)).

The construct 365-1451 (lacI/5′-lacZ no longer present) was found to be the best promoter and was used for all following integrations of reporter genes into the S. cerevisiae genome.

Primers Used:

365-for (SEQ ID NO 2)
ACGGGTACCGCAAAGGGAAGGGATGCTAAGG (KpnI)
394-for (SEQ ID NO 3)
ATCGGTACCTGAACGTTACAGAAAAGCAGG (KpnI)
1563-rev (SEQ ID NO 4)
ACTACTAGTGCCTCTTCGCTATTACGCCAGC (SpeI)
1674-rev (SEQ ID NO 5)
AGAACTAGTGGAAGATCGCACTCCAGC (SpeI)
1451-rev (SEQ ID NO 6)
ACAACTAGTAACTTTTCGGCCAATGGTCTTG (SpeI)
1366-rev (SEQ ID NO 7)
ACTACTAGTCCTATAGTTTTTTCTCCTGACGTTAAA (SpeI)

(ii) FOA treatment of S. cerevisiae Y190

The method of the invention requires reporter genes which are integrated into the genome of the yeast. This requires the availability of a selection marker so that only transformants that have actually integrated the desired gene at the correct locus in the genome can grow.

Thus the yeast strain Y190 needs an additional marker besides the auxotrophy markers leucine and tryptophan which are occupied by the two-hybrid system. Suitable herefor is Uracil (URA3 gene), since this gene offers the possibility of making the strain auxotrophic for said substance.

The preparation of URA3-negative clones makes use of the natural mutation frequency of yeast of about 10-4. In order to be able to select for the mutation events in the yeasts, a medium is used that contains FOA (5-fluoroorotic acid) (Treco DA, 1989). Yeast cells which no longer produce Uracil, i.e. which have the desired phenotype, survive, while the cells without mutation in the URA3 gene die (Boeke et al., 1984).

The colonies obtained in this process were checked for all markers before a yeast then serves as starting point of the following experiments (Y190D).

(iii) RedStar

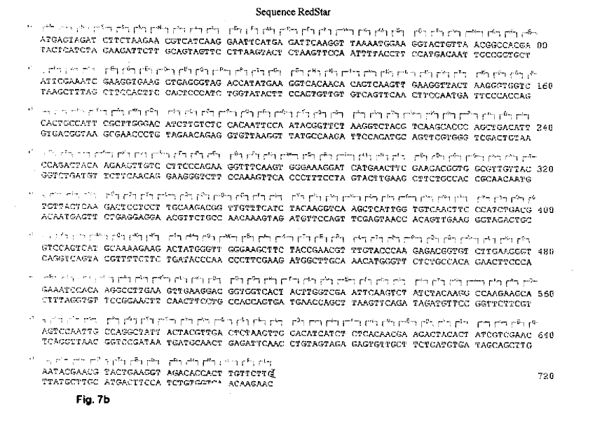

RedStar (RFP) is a fluorophore optimized for utilization in Saccharomyces cerevisiae, Knop et al., 2002. SEQ ID NO 8 (FIG. 7) represents the sequence of RedStar, comprising remnants of the cloning sequence. SEQ ID NO 34 (FIG. 7b) contains only the coding sequence.

For the GAL promoter and its amplification from the genome of Y190 see section (i).

RedStar was amplified using the following primers:

RedStar-for (SEQ ID NO 9)
ACTACTAGTTATGAGTAGATCTTCTAAGAACGTC (SpeI)
RedStar-rev (SEQ ID NO 10)
TATTCCGCGGTTACAAGAACAAGTGGTGTCTAC (SacII)

The particular promoter and the RedStar gene were cloned into pRS306 in a three-fragment ligation (25 fmol of vector, 125 fmol of inserts). pRS306 is an integration vector. Integration of RedStar (or any other reporter genes under the control of the GAL promoter) into the genome of the Uracil-auxotrophic yeast Y190D (see (ii)) can be selected for by means of the Uracil marker of pRS306.

### (iv) cob A

cob A codes for uroporphyrinogen III methyltransferase from Propionibacterium freudenreichii. Over expression of this gene results in a fluorescence of around 605 nm, which is due to accumulation of the fluorescent product trimethylpyrrocorphin (Wildt and Deuschle, 1999).

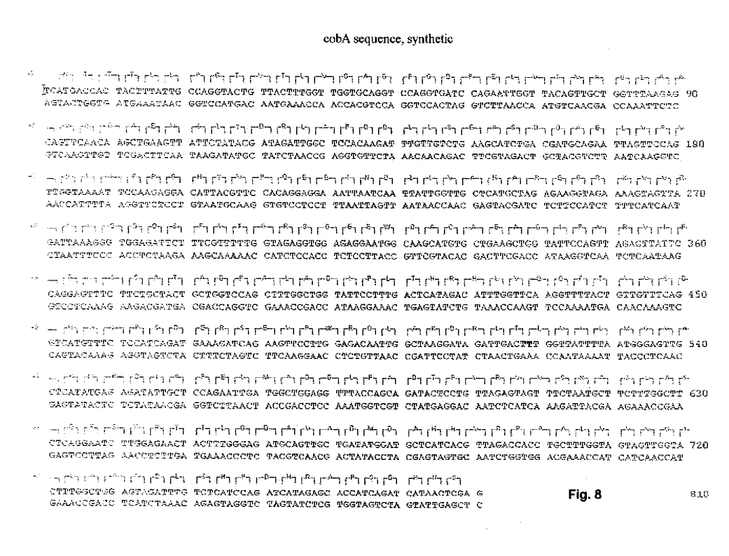

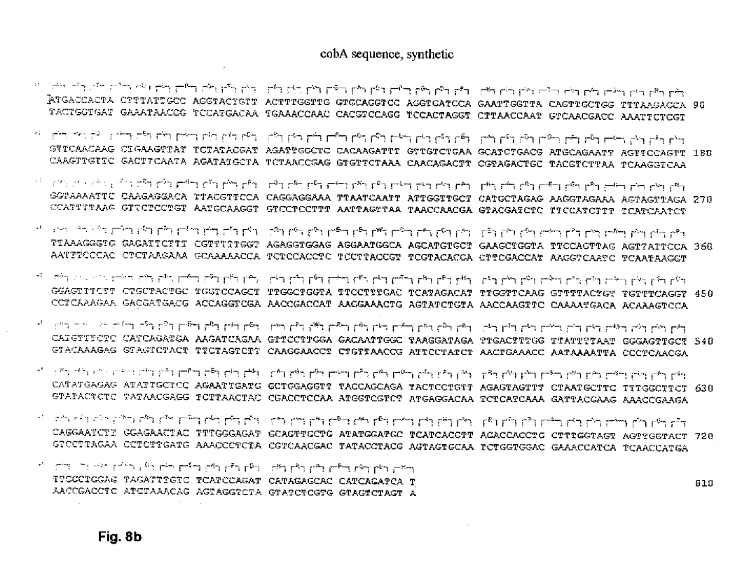

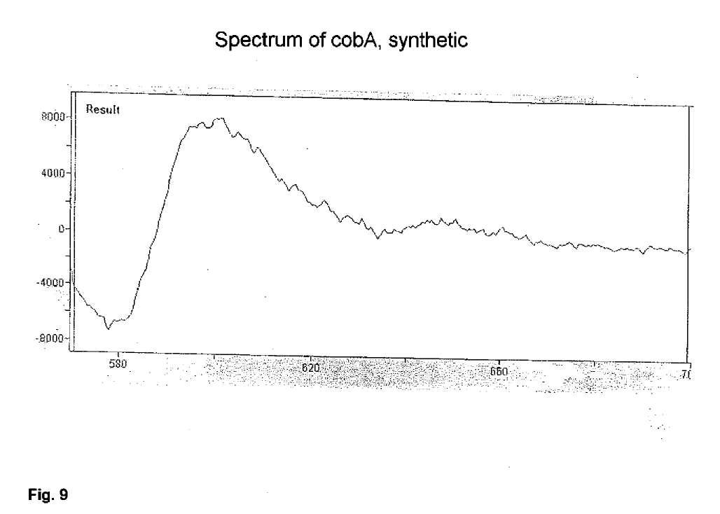

Analysis of the codon usage revealed a high percentage of critical codons for expression of the bacterial gene in yeast. Consequently, the sequence was optimized for the frequency of codon usage of S. cerevisiae and synthesized. FIG. 8 (SEQ ID NO 11) depicts the sequence of the codon-optimized DNA for cobA with remnants of the cloning site. FIG. 8b (SEQ ID NO 35) contains only the coding sequence.

In this case too, after attaching a His tag and a termination sequence (see above), the gene was cloned together with the promoter of choice (see above) via SacI/NotI into pRS306 and integrated into the genome of the yeast.

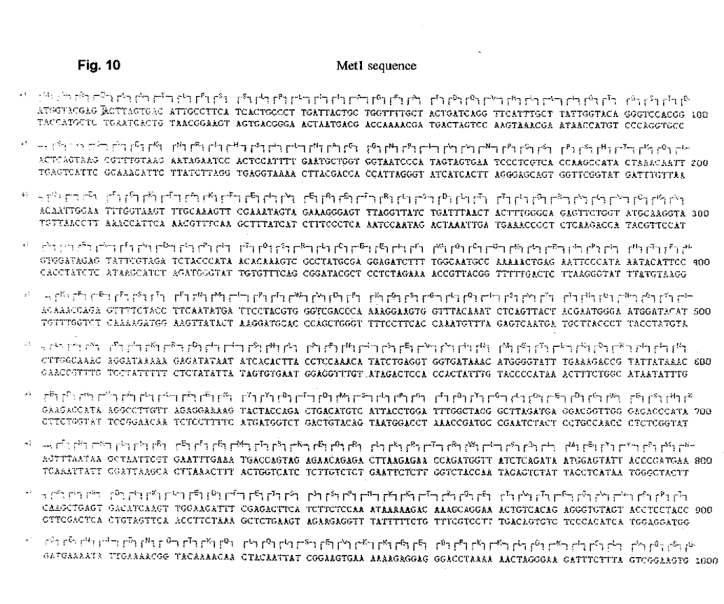

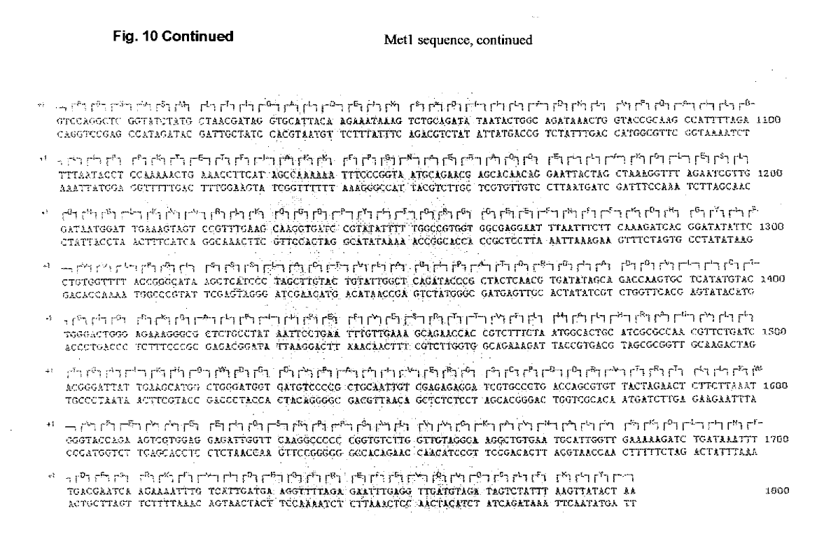

A distinct fluorescence of the yeast colonies is found. An emission spectrum recorded at the excitation wavelength of 540 nm for further validation proves the successful development with regard to the method of the invention (spectrum, see FIG. 9, difference spectrum of cobA-expressing yeast and yeast without additionally introduced reporter gene).

### (v) Met 1

Met1 is the corresponding Saccharomyces protein. The 1.8 kb gene was amplified from the yeast genome by means of PCR (SEQ ID NO 12 depicts the sequence including a few sections of the cloning sites; SEQ ID NO 36 depicts the coding sequence)

### Primers Used:

Met1-for (SEQ ID NO 13):
AATTATCCATGGTACGAGACTTAGTGACATTG
(NcoI)
Met-1-rev (SEQ ID NO 14):
AATTAACTCGAGTTGTATAACTTAAATAGACTATCTACATCAACC
(XhoI)

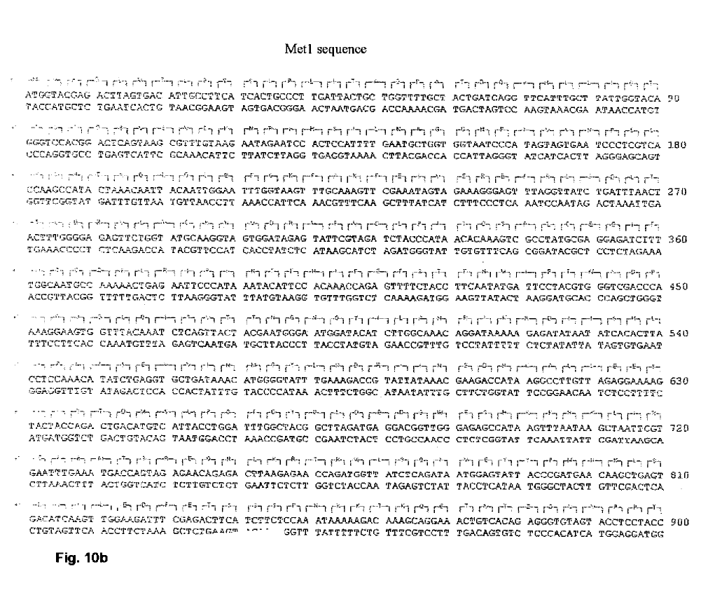

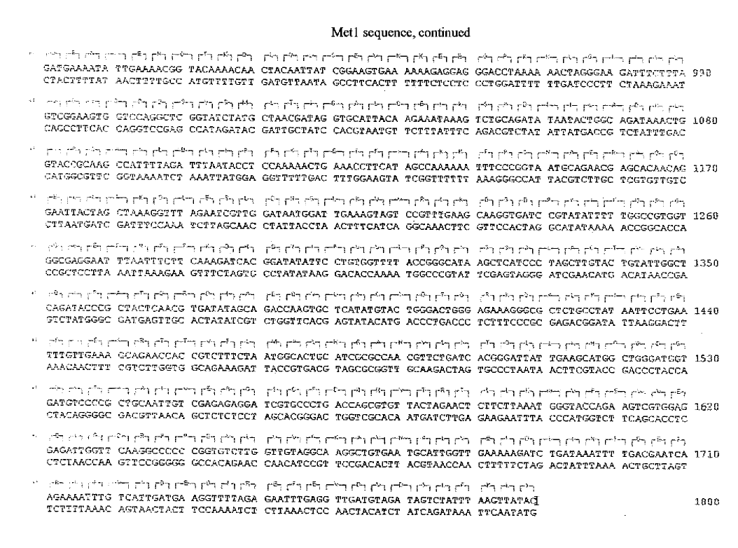

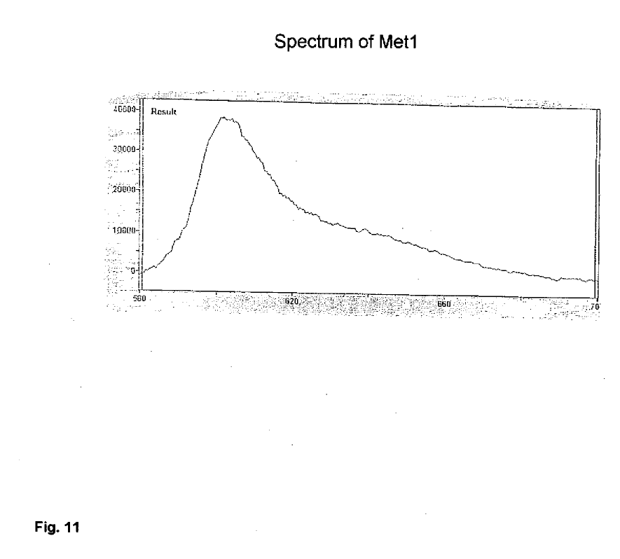

The fragment was cloned via NcoI/XhoI (NcoI contains the start codon) into a vector which enables a His tag and a termination sequence for yeast genes to be attached (Arntz et al., 2004). After cloning the reporter gene (NcoI/NotI) with the promoter of choice (SacI/NcoI) via SacI/NotI into the pRS306 vector, the reporter gene was integrated into the genome of the yeast. This reporter gene for the method of the invention was also successfully expressed. In the emission spectrum, at an excitation of 550 nm, the specific peak is largest at approx. 600 nm (see FIG. 11, difference spectrum of Met1-expressing yeast and yeast without additionally introduced reporter gene).

### b) Carrying Out Quantitative Screening in the Method of the Invention

Carrying out quantitative screening includes preparation of the medium, transformation of the yeasts and scanning of the plates. All parameters here need to be standardized and optimized in order for the fluorescence results to be reproducible.

### (vi) CysG^{A}

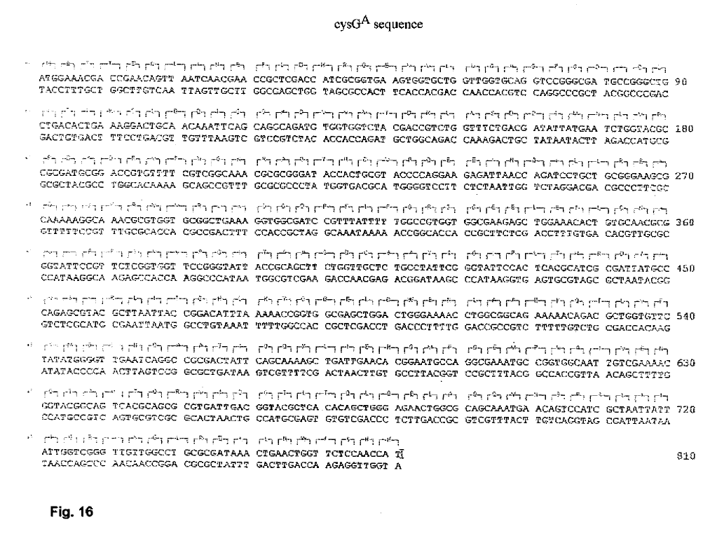

CysG^{A} comprises the C-terminal region (from amino acid 211) of CysG and thus the activity of UMT (Roessner, 2002). The 780 bp gene fragment (sequence, see FIG. 16 (SEQ ID NO 37) was amplified from the yeast genome by means of PCR.

### Primers Used:

| | |
|---|---|
| cysG-trunc-for: | |
| CCAACCCCATGGAAACGACCGAACAGTTAATC | (SEQ ID NO 38) |
| cysG-trunc-rev: | |
| AATGTTCTCGAGTTATGGTTGGAGAACCAGTTCAG | (SEQ ID NO 39) |

The fragment was cloned via NcoI/XhoI (NcoI contains the start codon) into a vector which enables a His tag and a termination sequence for yeast genes to be attached (Arntz et al., 2004). After cloning the reporter gene (NcoI/NotI) with the promoter of choice (SacI/NcoI) via SacI/NotL into the pRS306 vector, the reporter gene was integrated into the genome of the yeast.

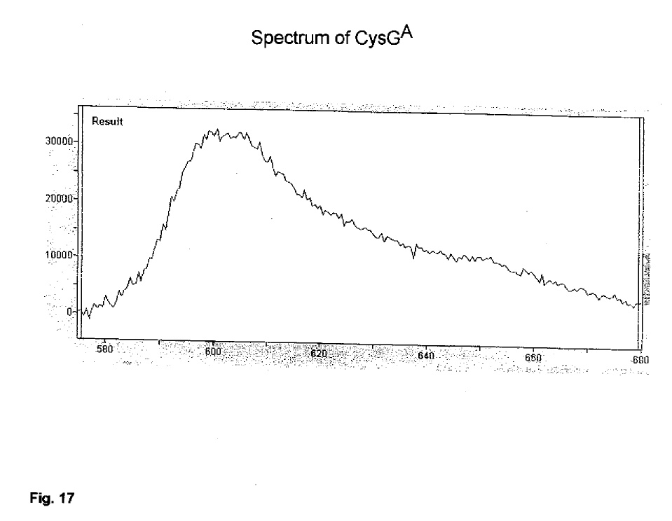

This reporter gene was also successfully expressed; in the emission spectrum, at an excitation of 545 nm, maximum emission is largest at approx. 605 nm (difference spectrum of CysG^{A}-expressing yeast and yeast without additionally introduced reporter gene, see FIG. 17).

### (ii) Preparation of the Medium

The following media are required for culturing and scanning the yeasts for fluorescence by means of the LSA scanner:

YPAD medium (complete medium for yeasts)
5.0 g of yeast extract (Difco)
10.0 g of peptone (Difco)
50 mg of adenine hemisulfate
ddH₂O ad 460 ml
Adjust pH to 5.8 prior to autoclaving; the medium has a pH of 5.6 after autoclaving;
For agar plates: addition of 10 g of yeast agar (Difco) after pH adjustment
Autoclaving for 15 minutes at 121° C.;
After autoclaving, 40 ml of 25% strength glucose (autoclaved separately from the medium) are added.
Synthetic Complete Medium (without Leu, Trp, His)
3.35 g of yeast nitrogen base (w/o amino acids)
1 g of synthetic complete drop out mix (amino acid mix without Leu, Trp, His)
ddH₂O ad 460 ml;
Adjust pH to 5.8 prior to autoclaving; the medium has a pH of 5.6 after autoclaving;
For agar plates: addition of 10 g of yeast agar (Difco) after pH adjustment
Autoclaving for 15 minutes at 121° C.;
After autoclaving, 40 ml of 25% strength glucose (autoclaved separately from the medium) and 10 ml of 2.5 M 3-amino-1,2,4-triazole (sterile-filtered) are added.
Final glucose concentration in the medium: 2%

25% Strength Glucose

100 g of glucose (Sigma)
400 ml of ddH₂O
Autoclaving for 15 minutes at 121° C.

2.5 M 3-amino-1,2,4-triazole

1.051 g of 3-amino-1,2,4-triazole
5 ml of ddH₂O
Filtering using a sterile filter (0.45 μm in diameter);
The final 3-amino-1,2,4-triazole concentration in the medium varies between 0 and 50 mM, depending on the experiment.

In a standard procedure, Omnitray plates (Nunc) on which the yeasts are cultured for scanning in the LSA scanner are poured, containing a volume of 78 ml of medium. This results in always the same scanning level, adjusted to 9.9 mm, for the scanner.

### (ii) Carrying Out Yeast Transformation

The following protocol is applied which has been optimized for the highest possible transformation efficiency.

20-30 ml of liquid YPAD (complete medium) are inoculated with the yeasts to be transformed (Y190D with integrated reporter gene) and incubated at 30° C. and 200 rpm overnight. On the next day, yeasts from the preculture are added by pipetting to 50 ml of YPAD (warmed to room temperature), until about 0.05 OD₆₀₀ is reached. The culture is incubated at 30° C. and 150-200 rpm, until a cell density of 2×10⁶−4×10⁶ cells/ml is reached. This corresponds to 0.2-0.4 OD₆₀₀ (takes approx. 3-5 h). The culture is harvested in a sterile 50 ml centrifuge tube at 3000×g (3500 rpm in a Hettich centrifuge) and 5 minutes. The medium (supernatant) is removed and the cells are resuspensed in 25 ml of sterile ddH₂O.

The cells are resuspended and then centrifuged again at 3000×g (3500 rpm, Hettich centrifuge) for 5 minutes. The supernatant is removed and the cells are resuspended in 1.0 ml of 100 mM lithium acetate. The suspension is transferred to a 1.5 ml Eppendorf cup. The cells are then incubated at 30° C. for 15 minutes. This is followed by pelleting the cells by centrifugation at “full speed” for 15 seconds and removing the supernatant by pipetting. This amount of cells is adequate for one transformation mixture. If two transformation mixtures are to be prepared, 100 ml (2×50 ml) of competent cells must be prepared and pretreated with lithium acetate. The following “transformation mix” is pipetted in the order indicated to the cells:

X μl of plasmid DNA (0.1-10 μg)

34-X μl of sterile ddH2O
(Resuspend cells in water+plasmid solution by pipetting up and down, only then at PEG by pipetting)
240 μl of PEG (50% w/v)
(Mix Cells with Peg by Vortexing Briefly)
36 μl of 1.0 M lithium acetate
50 μl of ss DNA (2.0 mg/ml)
Total volume: 360 μl
The cells are vortexed vigorously, until a homogeneous suspension is produced (approx. 1 min). The transformation mixture is incubated in a shaker (800 rpm) at 30° C. for 30 min and then placed in a waterbath at 42° C. (heat shock). After said incubations, the transformation mixture is centrifuged at 6-8000 rpm for 15 seconds and the transformation mix is removed from the Eppendorf cup using an Eppendorf pipette. The pellet (cells) is admixed with 1.0 ml of sterile ddH₂O and resuspended by pipetting slowly up and down. Pipetting rapidly up and down reduces transformation efficiency. The dissolved transformed cells are diluted once 1:100 and once 1:10 000, and from 2 to 200 μl of the diluted cells are straightened out on SC medium without leucine, tryptophan and histidine and with a suitable concentration of 3-aminotriazole. The number of colonies expected is 0-50 colonies per plate, with 20-200 μl of a 1:10 000 dilution being plated, and 200>5000 colonies per plate, with 10-200 μl of a 1:100 dilution being plated. The transformation efficiency then is 500 000-2 000 000 cells per μg of plasmid DNA (transformation efficiency decreases with increasing 3-AT concentration). The plates are incubated at 30° C. for 2-6 days.

After 2-6 days (depending on interacting pair and 3-AT concentration) the yeast cells can be scanned and evaluated in an LS-400 scanner (Tecan).

(iii) Tecan LS-400 Scanning

The hardware (Tecan LS-400 scanner) with matching software is available from Tecan.

The Agar level (scanning level) of the Omnitray plate is at least 8.0 mm.

For the measurements, established methods were applied. Clones whose genome contains Redstar, cobA or Met1 and which harbor 2-hybrid or n-hybrid plasmids are scanned using a 543 nm laser and a 590 nm filter (20 nm bandpass).

Recordings were carried out in a nonfocal manner.

The scan resolution (image resolution) is set to 20 μm when scanning a normally grown culture (diameter of approx. 1-2 mm). If a culture consists of smaller colonies, the scan resolution is reduced to from 4 to 8 μm.

### (iv) Evaluation of Scanned Colonies Using Optimate

The measurements are evaluated using the Optimate software which has been developed for this application in cooperation and is commercially available from Tecan.

The following settings are optimized for a culture consisting of colonies having a diameter of 1-2 mm:

### Minimum Object: 70

### Roundness: 15.2

### Threshold Power: 15

All colonies that are in an isolated position and are large enough are evaluated. The fluorescence intensity is normalized to the area.

### c) Cloning of the Competitor

### (i) Choosing Different Promoters for the Competitor—Preliminary Test

The concentration of the competitor is essential to the n-hybrid system. The more gene product is present, the more the equilibrium shifts to the side of the inactive complex of competitor and fusion bait protein.

To ensure variable concentrations of the competitor, different promoters described as constitutive in the literature (Nacken et al., 1996) are to be used for expressing the competitor and validated in our yeast strain.

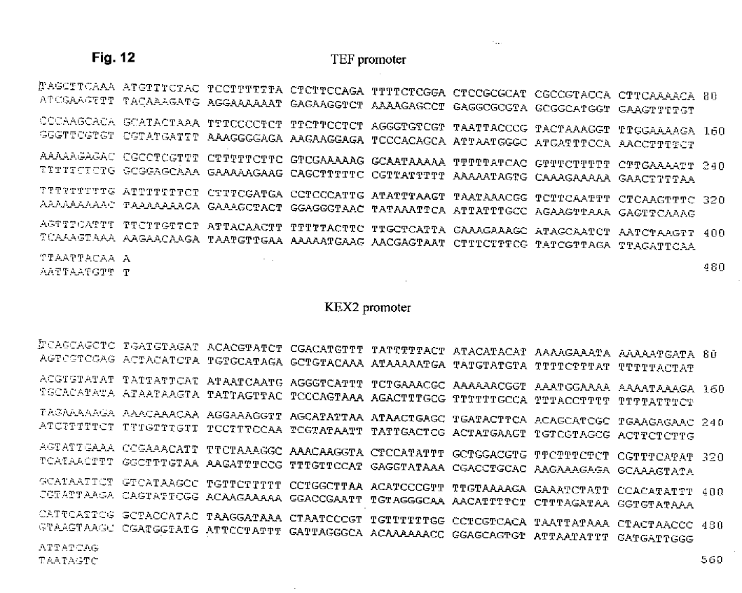

KEX2 (SEQ ID NO 17, Fuller et al., 1989, M24201), sequence, see FIG. 12, 488 bp

KEX2-for (SEQ ID NO 15):

ATCCTTGAGCTCTCAGCAGCTCTGATGTAGATACAC (SacI)

### KEX2-rev (SEQ ID NO 16):

ATCCCCCATGGCTGATAATGGGTTAGTAGTTTATAATTATGTG (NcoI)
TEF (SEQ ID NO 20, Cottrelle et al., 1985, M10992) sequence, see FIG. 12, 411 bp

### TEF-for (SEQ ID NO 18):

ATCCCCGCGGTAGCTTCAAAATGTTTCTACTCC (SacII)

### TEF-rev (SEQ ID NO 19):

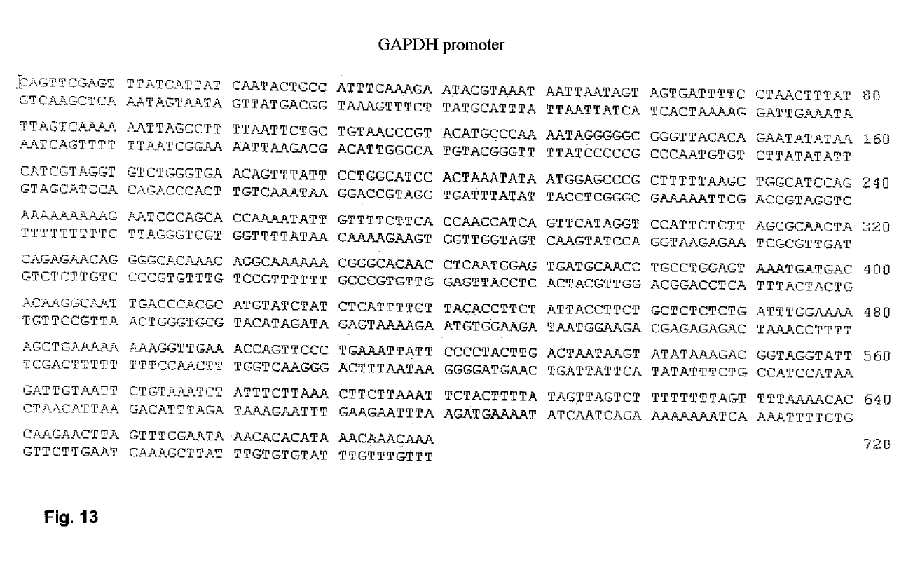

ATCCCCCATGGTTTGTAATTAAAACTTAGATTAGATTG (NcoI)
GAPDH (SEQ ID NO 23, Bitter and Egan, 1984 M13807): sequence, see FIG. 13, 680 bp

### GAPDH-for (SEQ ID NO 21):

ATCCCCGCGGCAGTTCGAGTTTATCATTATCAATAC (SacI)

### GAPDH-rev (SEQ ID NO 22):

ATCCCCCATGGTTGTTTGTTTATGTGTGTTTATTC (NocI)

These promoters were amplified from the yeast genome using the primers indicated (SacII or SacI/NcoI), cloned together with the RedStar gene (BspHI/NotI) into pRS306 (SacII or SacI/NotI) and integrated into the yeast genome. Determining the fluorescence intensity of the yeast colonies in the 2-hybrid system by means of quantitative screening resulted in the following order of promoter strength: GAPDH>TEF>KEX2; KEX2 can be called a very weak promoter. This result confirms the preliminary estimation according to the literature.

All three promoters were subsequently cloned upstream of the competitor (see the following section).

### (ii) Cloning of the Prey Protein Competitor to the Fusion Bait Protein Plasmid

For the method of the invention, the competitor is cloned into either of the two two-hybrid plasmids and thus ideally, like the bait and prey proteins, synthesized by the cell itself. If the prey protein is intended to be used as competitor, it is cloned to the vector containing the fusion bait protein; if the bait protein is intended to be the competitor, it is cloned to the vector containing the fusion prey protein. This prevents possible recombinations between identical gene sequences, which may take place in the yeast. An exemplary embodiment which will be described is the cloning of RafRBD (prey protein competitor) to pPC97 (fusion bait protein plasmid).

ppC₉₋₇-ras Contains the Following Structure:

### Promoter (ADH)-GAL4-BD-ras-mcs (AatII/SacI/SacII)-terminator (ADH)

In order to be able to clone the competitor, a terminator must be inserted downstream of the ras gene; then the promoter and then the competitor should follow. To this end, the terminator that is also attached to other genes to be cloned is used (see Arntz et al., 2004). In this case, two oligos are annealed (Term-Raf-for and -rev; sequence, see below). To this end, the oligos are annealed at a final concentration of in each case 2 μmol/μl in a PCR apparatus (94° C. 2 min, 70×−1° C., in each case 1 min at this temperature, 4° C.; information from Pierce: Anneal complementary pairs of oligonucleotides, Technical Resource). 2 μl are used for subsequent ligation into the vector.

Cloning into the fusion bait protein plasmid is carried out via AatII/SacI. The RafRBD gene is amplified (PciI/SacII) and cloned together with the particular promoter of choice (SacI/NcoI) into the vector with terminator in a three-fragment ligation (SacI/SacII). The result is the structure depicted in FIG. 5.

Primers Used:

Term-Raf-for (SEQ ID NO 24):
CTATATAACTCTGTAGAAATAAAGAGTATCATCTTTCAAAGAGCT
Term-Raf-rev (SEQ ID NO 25):
CTTTGAAAGATGATACTCTTTATTTCTACAGAGTTATATAGACGT
RafRBD-Pci-for (SEQ ID NO 26):
AATTCCACATGTCCGACCCGAGTAAGACAAGC (PciI)
RafRBD-SacII-rev (SEQ ID NO 27):
ATTGCCGCGGTTAGTCGACATCTAGAAAATCTACTTGAAG (SacII)

### 2. Limits of the Known Two-Hybrid System

The reporter gene activities of the RafRBD mutants, R67A, T68A, V69A and A85K, and of the wild type were investigated in the known two-hybrid system. These mutants are known to differ in their binding affinities and can be ordered according to increasing binding affinity as follows:

### RafRBD-R67A<T68A<V69A<WT<A85K

If these mutants are studied in the known two-hybrid system under the expression conditions described in Jaitner et al. (1997), this ranking is confirmed. In detail, the following values are found:

TABLE 1
Reporter gene activity using Met1 as reporter gene; reporter
gene activity is depicted as % of wild-type activity

| | Reporter fluorescence | Reporter activity in relation |
|---|---|---|
| RafRBD mutant | (arbitrary unit) | to wild type (WT) |
| RafRBD-R67A | 5196 | 62% |
| RafRBD-T68A | 5940 | 70% |
| RafRBD-V69A | 6975 | 83% |
| RafRBD-wt | 8430 | 100% |
| RafRBD-A85K | 9056 | 107% |

Therefore, comparison of the RafRBD-85K mutant with the wild type in the two-hybrid system leads to the conclusion that the mutant has an increase in binding activity by only 7%. However, it is known from microcalorimetric measurements that said mutant exhibits a distinctly higher affinity compared with the wild type, namely a dissociation constant of 72 nM compared to 253 nM of the wild type (see above and FIG. 4). This clearly indicates that the known two-hybrid system is not suitable for distinguishing high-affinity mutants from and identifying them via the wild type. That is because in practice a high-affinity protein can be identified only if the higher affinity of the mutated protein results in a clear and definite discrimination from the wild-type form by the readout of the method (here: reporter gene activity).

### 3. Influence of the Promoter on Determining the Reporter Gene Activity in the Method of the Invention with Competitor

The method of the invention may be varied, inter alia via the concentration of the competitor, in order to determine the dynamic range recorded in the study—i.e. to optimize the selection result. The concentration of the competitor expressed in the host cell can be controlled here, for example, by way of choosing the promoter upstream of the competitor (see 2c) cloning of the competitor).

The possibilities of influencing the system of the invention via the promoter of the competitor are demonstrated by experiments using the promoters of different strengths, KEX2, TEF and GAPDH. The weakest of these three promoters is KEX2, while GAPDH is the strongest (see 2c), cloning of the competitor).

Even using the weakest promoter (KEX2) for expressing the competitor results in a markedly improved distinction of the RafRBD-A85K mutant, whose affinity has been increased, from the wild-type protein. Thus, using Met1 as reporter, an activity of 120% (table 2) and, using RedStar as reporter, of 131% (table 3) compared to the wild-type form is measured. In comparison with the detectable activity of 107% in the conventional two-hybrid system (see above), this is indeed a basis on which in practice protein interactions with increased binding activity can be detected and consequently higher-affinity proteins can be identified.

TABLE 2
reporter gene activity using Met1 as reporter and with expression
of the competitor RafRBD-wt under the KEX2 promoter; reporter
gene activity is depicted as % of wild-type activity.

| | Reporter fluorescence | Reporter activity in |
|---|---|---|
| RafRBD mutant | (arbitrary unit) | relation to WT |
| RafRBD-R67A | 3264 | 41% |
| RafRBD-T68A | 5343 | 67% |
| RafRBD-V69A | 6651 | 83% |
| RafRBD-wt | 7969 | 100% |
| RafRBD-A85K | 9345 | 120% |

TABLE 3
reporter gene activity using RedStar as reporter and with expression
of the competitor RafRBD-wt under the KEX2 promoter; reporter
gene activity is depicted as % of wild-type activity.

| | Reporter fluorescence | Reporter activity in |
|---|---|---|
| RafRBD mutant | (arbitrary unit) | relation to WT |
| RafRBD-R67A | 4514 | 40% |
| RafRBD-T68A | 5737 | 51% |
| RafRBD-V69A | 7889 | 69% |
| RafRBD-wt | 11353 | 100% |
| RafRBD-A85K | 14857 | 131% |

When using the strong TEF promoter for expressing the competitor, the increase in RafRBD-A85K-induced Met1 reporter gene activity over the wild type is still further amplified (table 4). Thus, in the method of the invention using the TEF promoter, this reporter exhibits an activity which is at 137% compared to the wild type (using the KEX2 promoter, the activity was only 120%; table 2). If, in contrast, RedStar is used as reporter, the reporter gene activity is 139% compared to the wild type (table 5).

TABLE 4
reporter gene activity using Met1 as reporter and with expression
of the competitor RafRBD-wt under the TEF promoter; reporter
gene activity is depicted as % of wild-type activity.

| | Reporter fluorescence | Reporter activity in |
|---|---|---|
| RafRBD mutant | (arbitrary unit) | relation to WT |
| RafRBD-R67A | 1510 | 68% |
| RafRBD-T68A | 1600 | 72% |
| RafRBD-V69A | 1662 | 75% |
| RafRBD-wt | 2213 | 100% |
| RafRBD-A85K | 3031 | 137% |

TABLE 5
reporter gene activity using RedStar as reporter and with expression
of the competitor RafRBD-wt under the TEF promoter; reporter
gene activity is depicted as % of wild-type activity.

| | Reporter fluorescence | Reporter activity in |
|---|---|---|
| RafRBD mutant | (arbitrary unit) | relation to WT |
| RafRBD-R67A | 2747 | 30% |
| RafRBD-T68A | 4334 | 47% |
| RafRBD-V69A | 9095 | 99% |
| RafRBD-wt | 9233 | 100% |
| RafRBD-A85K | 12873 | 139% |

The method of the invention was also tested using the strong GAPDH promoter for expressing the competitor. Choosing this competitor, an increase in RedStar reporter gene activity compared to the wild type was again observed for the RafRBD-A85K variant. This activity was 168% (table 6).

TABLE 6
reporter gene activity using RedStar as reporter gene
and with expression of the competitor RafRBD-WT under
the control of the GAPDH promoter; reporter gene activity
is depicted as % of wild-type activity.

| | Reporter fluorescence | Reporter activity in |
|---|---|---|
| Construct | (arbitrary unit) | relation to WT |
| RafRBD-R67A | 1999 | 22% |
| RafRBD-T68A | 2997 | 34% |
| RafRBD-V69A | 7812 | 88% |
| RafRBD-wt | 8912 | 100% |
| RafRBD-A85K | 15014 | 168% |

### 4. Method of the Invention with Increased Selection Pressure

The relative reporter gene activity, recordable by the method of the invention, of the mutated prey or bait protein compared to the wild-type protein may still be increased by specific usage of a selection pressure on the transformed host cells (see above). Thus, for example, 3-aminotriazole can be added as inhibitor for His expression to the culturing medium.

In a particularly preferred embodiment, the method of the invention is carried out with expression of the competitor RafRBD-wt under the control of the TEF promoter and with addition of an increased aminotriazole concentration in comparison with the standard conditions described in Jaitner et al. (1997). This once more enhances discrimination between the wild-type protein and the affinity-improved RafRBD-A85K mutant. The activity of this mutant was 199% compared to the wild type (table 7).

TABLE 7
reporter gene activity using RedStar as reporter gene and
with expression of the competitor RafRBD-wt under the TEF
promoter as a function of 3-AT concentration; reporter gene
activity is depicted as % of wild-type activity.

| | Reporter activity in | Reporter activity in |
|---|---|---|
| | relation to the wild type | relation to the wild type |
| Construct | at 25 mM 3-AT | at 50 mM 3-AT |
| RafRBD-T68A | 21% | 34% |
| RafRBD-wt | 100% | 100% |
| RafRBD-A85K | 125% | 199% |

With the aid of the method of the invention it was therefore possible to unambiguously identify the RafRBD-A85K mutant, whose affinity is improved compared to Ras, on the basis of increased reporter gene activity.

### 5) Method of the Invention Using Random Mutagenesis and Robot-Assisted Hit Picking

To determine the functionality of the method of the invention, mutants with increased binding affinity must be able to be selected from a large number of foreign sequences. In this context, as explained, the method of the invention must have improved discrimination of the improved mutants from the wild type in comparison with the results of the two-hybrid method.

For this purpose, a method was carried out which is composed of generating the mutants (random mutagenesis), transforming the mutated vectors (“library”) into the yeast and hit picking which comprises selecting the most fluorescent colonies after quantitative screening by the robot (Tecan Genesis Freedom). This process is followed by isolating the plasmid DNA from the yeasts, transforming said DNA into bacteria (with both processes being robot-assisted), sequencing and finally evaluating the mutants obtained.

As an example of generating mutants with increased affinity, random mutageneses were carried out on the basis of the interacting pair Ras/RafRBD.

### a) RANDOM MUTAGENESIS

### Various Methods are Available for Random Mutagenesis (Neylon 2004).

Error Prone PCR (epPCR)

The advantages of epPCR are especially its universal usability and ready workability. In epPCR, as well as in the other methods in which copying of DNA is deliberately interfered with (e.g. use of mutator strains such as XL1-Red from stratagene and use of chemical and physical mutagens), the mutations are randomly distributed over the entire target gene. Methods are also described which mutagenize the entire plasmid at a certain rate (rolling circle amplification, Fujii et al. 2004). The error rate of the Taq polymerase used is increased, for example, by using Mn²⁺, unbalanced amounts of dNTPs or nucleoside triphosphate analogs (Zaccolo et al. 1996). Apart from these possibilities, two kits are offered which firstly are based on changes in Mn²⁺—and GTP concentrations (Diversify PCR Random Mutagenesis Kit, Clontech) and secondly use a highly error-prone polymerase and vary template concentration (GeneMorph, Stratagene).

epPCR as such is based either on inserting a wrong base and/or on the lack of proofreading ability of the polymerase. The inherent property of the polymerase used means that some errors appear more frequently than others. As a result, some mutations (such as, for example, transitions) appear more frequently than others, and the library is of a non-random nature (error bias). The bias of the libraries can be reduced by combining two different methods in which different biases occur, such as using the Taq polymerase and the GeneMorph kit.

There is furthermore the “codon bias” which is based on the nature of the genetic code. Simple point mutations result in a bias in the variants of amino acids encoded by the mutated DNA. For example, a point mutation in a valine codon produces only six different amino acids (Phe, Leu, Ile, Ala, Asp, Gly). In order to encode the other AAs, either two point mutations (C, S, P, H, R, N, T, M, E, Y) or even three point mutations (Q, W, K) are required.

The last bias is the “amplification bias”. It can be observed in any mutagenesis protocol that includes an amplification step. A molecule which has been copied early in the amplification process is over represented in the final library. This problem may, at least partially, be overcome by combining various, separately carried out epPCRs and/or by reducing the number of PCR cycles.

Another characteristic of epPCR is the fact that not all bases are accessible to mutagenization and that, from a statistical point of view, a given amino acid is mutagenized only to less than five other amino acids (Wong et al. 2004).

### Oligonucleotide-Based Methods

In contrast to epPCR in which a relatively long DNA sequence is mutagenized randomly, oligonucleotide-based methods have the aim of randomizing only individual, certain positions of the targeted gene. All techniques are based on incorporating into the coding sequence a synthetic DNA sequence (oligonucleotide) which may have been mutagenized to a different degree. Said DNA sequence may be one oligonucleotide or multiple primers at the same time.

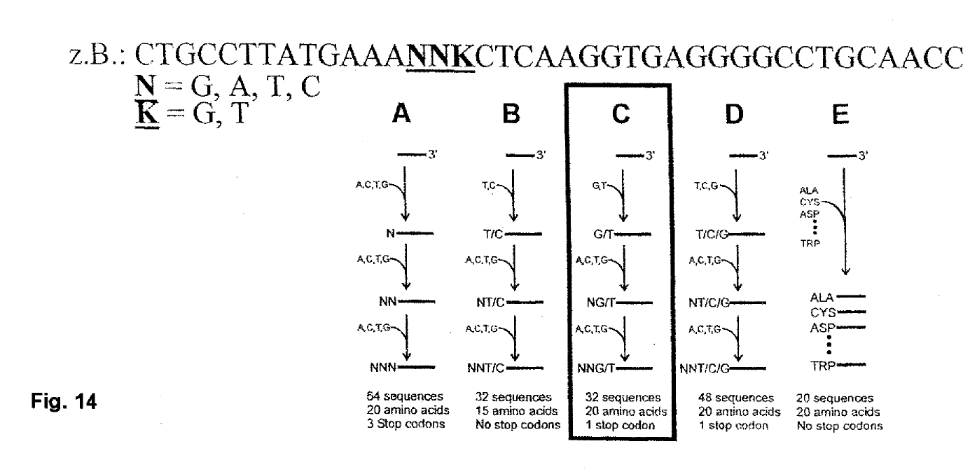

In order to encode all amino acids, different degeneses may be employed (see FIG. 14). Most frequently employed for the codon to be randomized is the combination NNK (N=G, A, T or C; K=G or T) because all AAs are encoded, the size of the library is only half the number of clones, compared to using NNN, and the probability for Met and Trp is 1/32, compared to 1164 with NNN.

The minimum number of clones containing all possible single mutants is defined by the frequency of the least represented mutants, i.e. the Ms which are encoded by only one codon (N, D, C, E, Q, H, I, K, M, F, W, Y), and the efficiency of the mutagenesis method employed. If an NNG/T codon is used, the frequency of the least represented mutant, (f) ¼×¼×½= 1/32. This means that, provided the mutation efficiency is 100%, approx. 100 clones must be screened in order to obtain all possible mutants with 95% probability ([0.95=1−(1−f)ⁿ]; n=number of clones screened).

The simultaneous insertion of two NNG/T codons gives (f): (¼×¼×½)²= 1/1024, thereby increasing the number of clones to be screened to 3100; with three NNG/T codons, the number is 10⁵ clones (calculations from Hogrefe et al. 2002).

Methods of incorporating oligonucleotides into the coding sequence can be divided into methods which allow mutations to be incorporated at various/multiple sites of the target DNA and techniques which are suitable especially for incorporating one or two mutagenic oligonucleotides.

The first category includes, for example, the methods ADO (assembly of designed oligonucleotides, Zha et al. 2003) and multiple-site-directed mutagenesis, described by Seyfang (2004). Zha et al. use overlapping oligonucleotides which anneal and are then amplified in a PCR. In Seyfang et al., oligonucleotides hybridize to ssDNA, followed by primer extension and ligation with likewise subsequent amplification of the mutated strand. Ness et al. (1995) also describe synthesis shuffling; these authors reconstruct a relatively large DNA region by means of overlapping oligos.

Hughes et al. (2003), with the “MAX method”, offer the possibility of carrying out a mutagenesis with defined oligos at multiple sites of the gene and in the process avoiding codon redundance, since each AA is represented by only one codon. The mutagenesis templates are randomized oligonucleotides; as a result, the length of the mutagenizable region is restricted, since long oligonucleotides may contain errors due to the synthesis. However, it might also be possible hero to anneal two (or more) oligonucleotides and assemble the entire gene in a primer extension reaction or overlap extension PCR.

Various methods are also available for incorporating one or a few oligonucleotides such as, for example, megaprimer techniques (Sarkar and Sommer 1990; variants and developments of Shepard and Rae, 1999; Tyagi et al. 2004), strand overlap extension (SOE, Higuchi et al. 1988) and QuikChange (Stratagene)-based methods. Hogrefe et al. (2002) make use of the QuikChange Multi Site-Directed Mutagenesis Kit with degenerated oligonucleotides as primers. Zheng et al. (2004) utilize only the principle of the QuikChange kit, but employ primers which overlap only partially, thus achieving a preference of the primers binding to the template over self pairing. The latter method is a simple and apparently efficient technique.

### b) ROBOT-ASSISTED PROCESS

On the principle of quantitative screening, see above

### (i) Hit Picking Using the Tecan Freedom 200

Hard- and software are commercially available from Tecan; the software was developed in cooperation.

The program Gemini runs the script “Colony-Pick” which comprises entering the number of hits to be picked in %. 70% ethanol is provided in the container “Steril 1” for sterilizing the pipetting and picking needles. The picked colonies are set down in microtiter plates containing the same selection medium (SC-LWH-Agar for selective yeast cultivation in 2-hybrid and N-hybrid), as the one on which the yeasts to be picked were cultured.

The process ColonyPicking is carried out by the software Facts; here a method of how to scan can be selected. Said method is defined for Colony-Pick (Gemini). That is, for clones containing the RedStar or the cobA gene as reporter gene, the “RedStar-Scanning” method must be carried out using the following settings:

### Scan Area Top 73 mm, Left 2 mm, Bottom 2 mm, Right 114 mm

### Autofocus: Z-Scan End 1600 μm, Z-Scan Start 1600 μm

### Focus Offset: 0 μm, Focal Plane: Plane 1

### Laser: 543 nm, Filter: 590 nm, Scan Resolution: 20 μm, Pinhole: Large

The Omnitray plates must be provided with a barcode.

After the colonies have been picked and cultured at 30° C. in an incubator for 2 days, the plasmids are reisolated from the yeasts and, after transformation into bacteria, sequenced.
(ii) DNA Isolation from Yeasts Using the Tecan T-Mags

After the yeasts have been cultured in the microtiter plates for 2 days, the DNA can then be isolated from the yeasts. 1000 μl of medium (SC-LWH) are pipetted into each well of a Deepwell plate. The colonies are then transferred from the resource plate (plate on which the yeast colonies grow after picking) to the Deepwell plate containing the respective selection medium. About 2001 of medium (SC-LWH)/well are added by pipetting to the yeasts in the microtiter plate, which are then resuspended by pipetting up and down several times. The resuspended yeasts are then transferred to the Deepwell plate to which medium has already been added previously. The yeasts are then incubated at 30° C. on a microtiter plate shaker for about 16 hours.

On the next day, the optical density of some wells is determined by adding 100 μl of these cells from a single well to 900 μl of medium. The optical density is then determined from this 10 fold dilution and then used for determining the average of all wells. The Deepwell plate containing the cells is then centrifuged in a swing-out rotor (Sorvall centrifuge) at 2500 rpm for 5 minutes. A second Deepwell plate filled with the same volume of H₂O is used as a counterweight. After centrifugation the supernatant is removed by decanting the Deepwell plate. 300 μl of Y1 buffer are pipetted into each well. In addition, 1-2 units of Lyticase/OD₆₀₀ (of the yeasts in the wells) are added by pipetting to each well. Buffer and Lyticase are mixed well with the cells. The Deepwell plate is incubated at 30° C. for 1.5 hours (no shaking). The Deepwell plate is centrifuged in a swing-out rotor at 2500 rpm for 5 minutes (Sorvall centrifuge). After centrifugation the supernatant is removed by decanting the Deepwell plate. The cells in the Deepwell plate are taken up in 250 μl of ddH₂O. The cells must be resuspended well. The Deepwell plate is then ready for DNA isolation.

Using the Gemini software of the Tecan Robot, a method of isolating the DNA is carried out, which has been developed by AGOWA (Berlin) together with Tecan. Said isolation takes place in the T-Mags apparatus on the robot platform.

(iii) Transformation of Bacteria

### Preparation of Competent Bacteria for Transformation in PCR Plates

5 ml of SOB medium are inoculated with a single colony of E. coli-DH5α bacteria. The cells are incubated at 37° C. on a shaker (210-225 rpm) overnight. 50-100 μl of this culture are transferred to 100 ml of SOB medium and incubated on a shaker (180 rpm) at 37° C. The bacteria are harvested at OD₆₀₀=0.1 to 0.5 (after approx. 2-3 hours) and placed on ice for 20 min. From hereon all further steps are carried out at a temperature of 4° C. The bacterial culture is centrifuged in a 50 ml Falcon vessel (conical bottom) at 4° C. and 1200 g. The pellet is resuspended by pipetting up and down in 10 ml of ice cold 50 mM CaCl₂ solution and then incubated on ice for at least 30 min. The cells are then centrifuged for 5 min at 4° C. and 1200 g. The cells are resuspended by pipetting up and down in 1 ml/0.1 OD₆₀₀ ice cold 50 mM CaCl₂ solution containing 15% glycerol. Thus the cells are taken up in 1 ml of CaCl₂ at OD₆₀₀=0.1 and in 3 ml of CaCl₂ at OD₆₀₀=0.3. 10 μl aliquots per well are introduced to a PCR plate precooled on ice and frozen and stored at −80° C.

Transformation of Bacteria with the DNA from Yeasts

The competent bacteria in the PCR plates are thawed in a metal PCR block placed on ice. To each well 10 μl of isolated DNA are added by pipetting. Bacteria and DNA are carefully mixed (no pipetting up and down!). The cells are then incubated on ice for at least 30 min. Subsequently a 30 s heat shock is carried out at 42° C. on a heating block. After the heat shock the cells are placed again on ice for 2 min. 100 μl of SOC medium are introduced into a Deepwell plate. Likewise, 100 μl of SOC per well are also added to the bacteria by pipetting. The bacteria are transferred from the PCR plate to the Deepwell plate and incubated on a microtiter plate shaker at 210 to 225 rpm at 37° C. for 1 hour. After 1 hour of incubation, 1 ml of LB medium containing the appropriate antibiotic is added to the cells by pipetting and incubated on a microtiter plate shaker at 210 to 225 rpm at 37° C. for at least 20 hours. After this incubation 5-10 μl of the cells are transferred to a 96-well plate containing LB agar+antibiotic, and said plate is incubated in an incubator at 37° C. for 16 hours. This plate can be sent to AGOWA for sequencing of the individual colonies.

### c) EXEMPLARY EMBODIMENT

### Mutagenesis on RafRBD A85

### (i) Construction of the Required Vectors

After transformation of the mutagenized library, two plasmids are present in the yeast, both of which carry an ampicillin resistance gene. Firstly, pPC97 containing the Ras gene (in the method of the invention this plasmid contains in addition also the competitor) and secondly, pPC86 which encodes the mutated RafRBD gene. After transformation of the DNA isolated from said yeasts into competent bacteria, only the plasmid containing the mutated RafRBD gene should still be present in the bacteria. For this purpose, one of the vectors must be equipped with a different antibiotic resistance. In the present case, pPC86 was provided with a canamycin resistance. To this end, a PmeI site was generated in each case upstream and downstream of the TEM resistance gene by means of QuikChange mutation according to the manufacturer's information, the gene was subsequently excised and replaced with the canamycin resistance gene. The bacteria transformed with the DNA from yeast now grow in medium containing canamycin and can in this way be separated from the bacteria containing the fusion bait protein plasmid, pPC97.

Primers Used:

Multi-QC-Pme-vor-TEM (SEQ ID NO 28)
TGAATACTCATACTCTTCCTGTTTAAACATTATTGAAGCATTTATCAGGG
Multi-QC-Pme-nach-TEM (SEQ ID NO 29):
TTAAATCAATCTAAAGTATATATGTTTAAACTTGGTCTGACAGTTACCAA
TG (PmeI)
Pme-Kan-for (SEQ ID NO 30):
AAAAAACCGTTTAAACAGGAAGAGTATGATTCAACAAGATGGATTGC
(PmeI)
Pme-Kan-rev (SEQ ID NO 31):
AAAAAACCGTTTAAACTTGGTCTGACAGTCAGAAGAACTCGTCAAGAAGG
(PmeI)

### (ii) Random Mutagenesis Procedure

Random mutagenesis is carried out according to the method of Zheng et al. (2004) (see section b). To this end, the following two primers were designed which partially overlap and randomize the amino acid A85 of RafRBD:

Z-RBD-A85-forl (SEQ ID NO 32):
CTGCCTTATGAAANNKCTCAAGGTGAGGGGCCTGCAACCAG
Z-RBD-A85-rev (SEQ ID NO 33):
CCCTCACCTTGAGMNNTTTCATAAGGCAGTCATGCAAGCTC

These primers are used for a PCR using the Expand Kit (Roche). For this, 50 ng of template DNA (pPC86-RafRBD with canamycin resistance gene) are used and the PCR is carried out using 0.8 pmol/μl of each primer according to the manufacturer's information. The PCR reaction is then purified using the PCR purification kit (Qiagen), and 5 out of 50 μl are applied to an agarose gel. The remaining mixture is restricted with 10 U of DpnI (NEB, in buffer 4) at 37° C. for 3 hours, in order to remove the methylated, due to isolation from E. coli, template DNA. Zheng et al., at this point, carried out the digestion for only 1 hour, but this resulted in a high background of wild-type clones in the library.

Subsequently, 2.5 μl of the DpnI cut are transformed into 75 μl of competent XL10 Gold cells (Stratagene) according to the manufacturer's information; addition of 750 μl of NZY after the heat shock is followed by a 1 hour bacteria regeneration phase. 20 μl of the transformation mixture (approx. 1/20 of the total mixture) are plated out to determine the transformation efficiency, and the remainder is incubated in LB medium containing canamycin at 37° C. and 225 rpm on a shaker overnight. The DNA is isolated the next morning. The number of probably independent colonies is determined by counting the plated-out colonies and projecting the result to the total number (factor of 20). This number is then divided by 4 because the bacteria are assumed to divide twice during the 1 hour regeneration period. This value should be markedly above 100 for a representative library to be assumed (see calculations in the random mutagenesis section). In the present case, 152 colonies were counted after transformation, meaning a number of approx. 760 independent colonies in the mixture.

The library is characterized by sequencing individual colonies and the library DNA. This DNA is then transformed into yeast whose genome contains RedStar, Met1 or cobA as reporter gene. The second plasmid used here is either pPC9-7-ras (for the two-hybrid system) or pPC9-7-ras with TEF promoter/RafRBD competitor (for the n-hybrid system, methods of the invention).

(iii) Generation of Fragments for the Use of Homologous Recombination in the Method of the Invention

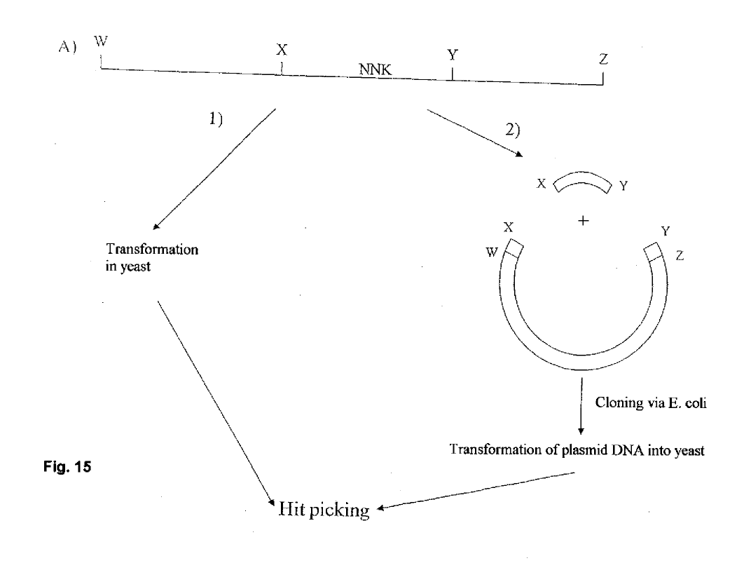

For this purpose, part of the library DNA generated under (ii) was restricted using the enzymes XmaI (W in FIG. 15) and SalI (Z in FIG. 15). The fragments resulting therefrom were eluted from the gene. They carry NNK at position 85 of the RafRBD gene and overlap to 90 bp at the 5′ end and to 70 bp at the 3′ end with the in each case corresponding ends of the vector. The latter was cleaved with BstBI (X in FIG. 15) and AscI (Y in FIG. 15).

### (iv) Robot-Assisted Process

Quantitative screening is carried out for yeast colonies which had grown on medium containing 50 mM 3-AT. Hit picking, DNA isolation and bacteria transformation are carried out as described above.

(v) Sequencing and Comparative Evaluation of Hits in the Two-Hybrid System and in the Method of the Invention (n-Hybrid System).

Results of the sequencing of the DNA from the bacteria colonies are depicted below. Both the two-hybrid system and the method of the invention were carried out several times. In this involved choosing both different time points over a relatively long period of time and different DNA preparations and fluorophores in order to prove the reproducibility and general validity of the results.

Since the mutants P (Proline), G (Glycine) and S (Serine) were, after the wild type, the next most common amino acids detected in the two-hybrid system, these amino acids have also been included in the tables for the method of the invention. The amino acids found in addition to the amino acids mentioned are denoted “other”.

### Use of Circular Vectors

### RedStar Fluorophore

Hit Picking Results Using the Two-Hybrid System

TABLE 8
result of hit picking using the two-hybrid
system with RedStar as reporter gene
a)

| | Two-hybrid | | Two-hybrid | | Two-hybrid | |
|---|---|---|---|---|---|---|
| | 39 colonies | | 33 colonies | | 29 colonies | |
| Encoded AA | Number | % | Number | % | Number | % |
| K (Lys) | 4 | 10.2 | 9 | 27.3 | 1 | 3.4 |
| R (Arg) | 12 | 30.7 | 5 | 15.2 | 4 | 13.8 |
| A (Ala) | 1 | 2.5 | 7 | 21.2 | 7 | 24.1 |
| P (Pro) | 6 | 15.3 | 4 | 12.1 | 6 | 20.7 |
| G (Gly) | 6 | 15.3 | 3 | 9.1 | 4 | 13.8 |
| S (Ser) | 7 | 17.9 | | | 3 | 10.3 |
| Other | 3 | 7.6 | 5 | 15.2 | 4 | 13.8 |

b)

| | Two-hybrid | | Two-hybrid | | Two-hybrid | |
|---|---|---|---|---|---|---|
| | 26 colonies | | 14 colonies | | 15 colonies | |
| Encoded AA | Number | % | Number | % | Number | % |
| K (Lys) | 5 | 19.2 | 3 | 21.4 | 2 | 13.3 |
| R (Arg) | 6 | 23.1 | 4 | 28.6 | 2 | 13.3 |
| A (Ala) | 8 | 30.8 | 2 | 14.3 | 4 | 26.7 |
| P (Pro) | 1 | 3.8 | | | 1 | 6.7 |
| G (Gly) | 3 | 11.5 | 3 | 21.4 | 2 | 13.3 |
| S (Ser) | 1 | 3.8 | 1 | 7.1 | 2 | 13.3 |
| Other | 2 | 7.7 | 1 | 7.1 | 2 | 13.3 |

Hit Picking Results Using the Method of the Invention

TABLE 9
result of hit picking using RedStar as reporter gene
and with expression of the RafRBD-wt competitor under
the control of the TEF promoter (n-hybrid system)
a)

| | n-hybrid | | n-hybrid | | n-hybrid | |
|---|---|---|---|---|---|---|
| | 32 colonies | | 18 colonies | | 41 colonies | |
| Encoded AA | Number | % | Number | % | Number | % |
| K (Lys) | 6 | 18.8 | 12 | 66.7 | 29 | 70.7 |
| R (Arg) | 19 | 59.4 | 3 | 16.7 | 9 | 21.4 |
| A (Ala) | | | | | 1 | 2.4 |
| P (Pro) | 4 | 12.5 | 2 | 11.1 | 1 | 2.4 |
| G (Gly) | 2 | 6.3 | | | 1 | 2.4 |
| S (Ser) | 1 | 3.1 | 1 | 5.6 | | |
| Other | | | | | | |

b)

| | n-hybrid | | n-hybrid | | n-hybrid | |
|---|---|---|---|---|---|---|
| | 52 colonies | | 34 colonies | | 17 colonies | |
| Encoded AA | Number | % | Number | % | Number | % |
| K (Lys) | 24 | 46.2 | 17 | 50.0 | 11 | 64.7 |
| R (Arg) | 19 | 36.5 | 15 | 44.1 | 5 | 29.4 |
| A (Ala) | 2 | 3.8 | | | | |
| P (Pro) | 3 | 5.8 | | | | |
| G (Gly) | 2 | 3.8 | 1 | 2.9 | | |
| S (Ser) | 1 | 1.9 | | | | |
| Other | 1 | 1.9 | 1 | 2.9 | 1 | 5.9 |

cobA Reporter Gene

Hit Picking Results

TABLE 10
result of hit picking using cobA as reporter gene and without
(two-hybrid system) or with expression of the RafRBD-wt competitor
under the control of the TEF promoter (n-hybrid system)

| | |
|---|---|
| Two-hybrid | n-hybrid |
| 34 colonies | 27 colonies |

| Encoded AA | Number | % | Number | % |
|---|---|---|---|---|
| K (Lys) | 7 | 20.6 | 8 | 29.6 |
| R (Arg) | 13 | 38.2 | 16 | 59.3 |
| A (Ala) | 3 | 8.8 | | |
| P (Pro) | 3 | 8.8 | 1 | 3.7 |
| G (Gly) | 3 | 8.8 | 1 | 3.7 |
| S (Ser) | 2 | 5.9 | 1 | 3.7 |
| Other | 3 | 8.8 | | |

### Met1 Reporter Gene

Hit Picking Results Using the Two-Hybrid System

TABLE 11
result of hit picking using the two-hybrid
system with Met1 as reporter gene

| | |
|---|---|
| Two-hybrid | Two-hybrid |
| 19 colonies | 22 colonies |

| Hit | Number | % | Number | % |
|---|---|---|---|---|
| K | 7 | 36.8 | 8 | 36.4 |
| R | 4 | 21.1 | 6 | 27.3 |
| A | 1 | 5.3 | | |
| P | 2 | 10.5 | | |
| G | 2 | 10.5 | 6 | 27.3 |
| S | 3 | 15.8 | 2 | 9.1 |
| Other | | | | |

Hit Picking Results Using the Method of the Invention

TABLE 12
result of hit picking using Met1 as reporter gene and
with expression of the RafRBD-wt competitor under the
control of the TEF promoter (n-hybrid system)

| | |
|---|---|
| n-hybrid | n-hybrid |
| 60 colonies | 44 colonies |

| Hit | Number | % | Number | % |
|---|---|---|---|---|
| K | 39 | 65.0 | 28 | 63.6 |
| R | 17 | 28.3 | 13 | 29.5 |
| A | 1 | 1.7 | | |
| P | 2 | 3.3 | | |
| G | | | 3 | 6.8 |
| S | | | | |
| Other | 1 | 1.7 | | |

### Use of Homologous Recombination

### Redstar Fluorophore

Hit Picking Results Using the Two-Hybrid System

TABLE 13
Result of hit picking using the two-hybrid
system with RedStar as reporter gene

| | Two-hybrid homologous | | Two-hybrid homologous | |
|---|---|---|---|---|
| | recombination | | recombination | |
| | 31 colonies | | 40 colonies | |
| Encoded AA | Number | % | Number | % |
| K (Lys) | 6 | 19.4 | 10 | 25 |
| R (Arg) | 9 | 29 | 13 | 32.5 |
| A (Ala) | 3 | 9.7 | 7 | 17.5 |
| P (Pro) | 4 | 12.9 | 5 | 12.5 |
| G (Gly) | 5 | 16.1 | 2 | 5 |
| S (Ser) | 2 | 6.5 | 2 | 5 |
| Other | 2 | 6.5 | 1 | 2.5 |

Hit Picking Results Using the Method of the Invention

TABLE 14
Result of hit picking using RedStar as reporter gene
and with expression of the RafRBD-wt competitor under
the control of the TEF promoter (n-hybrid system)

| | n-hybrid homologous | | n-hybrid homologous | |
|---|---|---|---|---|
| | recombination | | recombination | |
| | 42 colonies | | 40 colonies | |
| Encoded AA | Number | % | Number | % |
| K (Lys) | 9 | 21.4 | 18 | 45.0 |
| R (Arg) | 30 | 71.4 | 19 | 47.5 |
| A (Ala) | | | 2 | 5.0 |
| P (Pro) | 3 | 7.1 | | |
| G (Gly) | | | 1 | 2.5 |
| S (Ser) | | | | |
| Other | | | | |

Tables 8 to 14 reveal that it is very well possible to discriminate with the aid of the method of the invention between the improved mutant A85K (Fridman et al., 2000) described in the literature and the wild type. Wild type (A=alanine) was detected in tiny numbers using the method of the invention. In contrast, distinctly more wild type has been picked in the known two-hybrid system which used the same DNA containing the randomized position. The improved mutants K (Lysine) and R (Arginine, Fridman et al., 2000) are found in substantially smaller numbers in the two-hybrid system. This result again confirms the above-described difficulties (see table 1) in discriminating between A85K and the wild type in the two-hybrid system according to the prior art.

The number of amino acids detected apart from the wild type and the improved mutants is also substantially reduced in the n-hybrid system.

The data obtained using homologous recombination fully confirm the comments made; it is also possible to use the method of the invention and homologous recombination at the same time.
These data demonstrate the clear superiority of the method of the invention over the two-hybrid system. Therefore the object of the invention, to extend the dynamic range by using a competitor, has been achieved.

### REFERENCES

Arntz C., Meinders D., Block C., Mittmann K. (2004) Phycocyanin exprimierende Eukaryontenzelle [Phycocyanine-expressing eukaryotic cell], EPA 04 001 504.2
Aronheim A., Zandi E., Elledge S. J. and Karin M. (1997) Isolation of an AP-1 Repressor by a novel Method for detecting Protein-Protein Interactions. Mol. Cell. Biol. 17, 3094-3102
Bitter G. A. and Egan K. M. (1984) Expression of heterologous genes in Saccharomyces cerevisiae from vectors utilizing the glyceraldehydes-3-phosphate dehydrogenase gene promoter. Gene 32: 263-274
Boeke J. D., LaCroute F., Fink G. R. (1984) A positive selection for mutants lacking orotidine-5′-phosphate decarboxylase activity in yeast: 5-fluoro-orotic acid resistance Mol. Gen. Genet. 197(2); 345-6
Botstein D., Falco S. et al. (1979) Sterile host yeasts (SHY): A eukaryotic system of biological containment for recombinant DNA experiments. Gene 8: 17-24
Bulter T. and Alcalde M. (2003) Preparing Libraries in Saccharomyces cerevisiae. Methods in Molecular Biology 231: 17-22, Directed Evolution Library Creation: Methods and Protocols, Humana Press
Chevray P. M., Nathans D. (1992) Protein interaction cloning in yeast: identification of mammalian proteins that react with the leucine zipper of Jun. PNAS 89(13); 5789-5793
Cottrelle P., Thiele D., Price V., Memet S., Micouin J-Y., Marck C., Buhler J-M., Sentenac A., Fromageot P. (1985) Cloning, Nucleotide Sequence, and Expression of One of Two Genes Coding for Yeast Elonation Factor 1a. JBC 260: 3090-3096
de Felipe, K. S., Carter B. T., Althoff, E. A. and Cornish, V. W. (2004) Correlation between Ligand-Receptor Affinity and the Transkription Readout in a Yeast Three-Hybrid System. Biochemistry 43, 10535-10363
Estojak, J., Brent R. and Golemis E. (1995) Mol. cell Biol. 15, 5820-5829
Fields S., Song O. (1989) A novel genetic system to detect protein-protein interactions. Nature 340, 245-246
Fridman M., Maruta H., Gonez J., Walker F., Treutlein H., Zeng J. and Burgess A. (2000) Point Mutants of c-raf-1 RBD with elevated Binding to v-Ha-Ras. J. Biol. Chem. 275,
Fujii R., Kitaoka M. Hayashi K. (2004) One-step random mutagenesis by error-prone rolling circle amplification. Nucleic Acids Research 32(19) e145
Fuller R. S., Brake A., Thorner J. (1989) Yeast prohormone processing enzyme (KEX2 gene product) is a Ca²⁺-dependent serine protease. PNAS 86: 1434-1438
Guarente L., Ptashne M. (1981) Fusion of Escherichia coli lacZ to the cytochrome c gene of Saccharomyces cerevisiae. PNAS 78: 2199-2203
Higuchi T., Krummel B., Saiki R. K. (1988) A general method for in vitro preparation and specific mutagenesis of DNA fragments: study of protein and DNA interactions. Nucleic Acids Research 16: 7351-7367
Hogrefe H. H., Cline J., Youngblood G. L., Allen R. M. (2002) Creating Randomized Amino Acid Libraries with the QuikChange Multi Site-Directed Mutagenesis Kit. BioTechniques 33(5): 1158-1165
Hua S-B., Qiu M., Chan E., Zhu L., Luo Y. (1997) Minimum Length of Sequence Homology Required for in Vivo Cloning by Homologous Recombination in Yeast. Plasmid 38: 91-96
Hughes M. D., Nagel D. A., Santos A. F., Sutherland A. J., Hine A. V. (2003) Removing the Redundancy From Randomised Gene Libraries. J. Mol. Biol. 331: 973-979
Jaitner B. K., Becker J., Linnemann T., Herrmann C., Wittinghofer A. and Block C. (1997) Discrimination of Amino Acids mediating Ras binding from noninteracting Residues affecting Raf Activation by Double Mutant Analysis. J. Biol. Chem. 272, 29927-29933
Knop M., Barr F., Riedel C. G., Heckel T., Reichel C. (2002) Improbed Version of the Red Fluorescent Protein (drFP583/DsRed/RFP). BioTechniques 33: 592-602
Knop M., Barr F., Riedel C. G., Heckel T. and Reichel C. (2002) Improved version of the red fluorescent protein (drFP583/DsRed/RFP). Biotechniques 33, 592-602.
Leuther K. K., Johnston S. A. (1992) Nondissociation of GAL4 and GAL80 in Vivo After Galactose Induction. Science 256:1333-1335
Nacken V., Achstetter T., Degryse E. (1996) Probing the limits of expression levels by varying promoter strength and plasmid copy number in Saccharomyces cerevisiae Gene 175: 2563-260
Ness J. E., Kim S., Gottman A. et al. (1995) Synthetic shuffling expands functional protein diversity by allowing amino acids to recombine independently. Nature Biotechnology 20:
Neylon C. (2004) Chemical and biochemical strategies for the randomization of protein encoding DNA sequences: library construction methods for directed evolution. Nucleic Acids Research 32(4): 1448-1459
Osborne M. A., Dalton S, and Kochan J. P. (1995) The Yeast Tribrid System-Genetic Detection of trans-phosphorylated ITAM=SH2-interactions. Bio/Technology 13, 1474-1478
Roessner C. A. (2002) Use of cobA and cysGA as red fluorescent indicators. Methods Mol. Biol. 183, 19-30
Roessner C. A. and Scott A. I. (1995) Fluorescence-based method for selection of recombinant plasmids. BioTechniques 19: 760-764
Sandrock B. and Egly J. M. (2001) A Yeast Four-Hybrid System identifies CDK-activating Kinase as a Regulator of the XPD Helicase, a Subunit of Transcription Factor IIH. J. Biol. Chem. 276, 35328-35333
Sarkar G. and Sommer S. S. (1990) The “megaprimer” method of site-directed mutagenesis. BioTechniques 8: 404-407
Seizer T., Albeck S, and Schreiber G. (2000) Rational design of faster associating and tighter binding complexes. Nature Struct. Biol. 7, 537-541
Serebriiskii I., Khazak V. and Golemis E. A. (1999) A Two-Hybrid dual Bait System to discriminate Specificity of Protein Interactions. J. Biol. Chem. 274, 17080-17087
Seyfang A. and Jin J. H. (2004) Multiple site-directed mutagenesis of more than 10 sites simultaneously and in a single round. Analytical Biochemistry 324: 285-291
Shepard A. R. and Rae J. L. (1999) Simple Version of “Megaprimer” PCR for Site-Directed Mutagenesis. BioTechniques 26: 870-873
Tirode F., Malaguti C., Romero F., Attar F., Camonis J. and Egly J. M. (1997) A conditionally expressed third Partner stabilizes or prevents the Formation of a Transcriptional Activator in a Three-Hybrid System. J. Biol. Chem. 272, 22995-22999
Treco D. A. Basic Techniques of Yeast Genetics (1989) in: Current Protocols in Molecular Biology, Wiley, Supplement 5, Unit 13.1, p. 13.1.5.
Tyagi R., Lai R., Duggleby R. G. (2004) A new approach to “megaprimer” polymerase chain reaction mutagenesis without an intermediate gel purification step. BMC Biotechnology 4: 2-7
Vidal M., Brachmann R. K., Fattaey A., Harlow E. and Boeke J. (1996) Reverse Two-Hybrid and One-Hybrid Systems to detect dissociation of protein-protein and DNA-protein interactions. Proc. Natl. Acad. Sci. USA 93, 10315-10320
West R. W., Yocum R. R. Ptashne M. (1984) Saccharomyces cerevisiae GAL-1-GAL10 Divergent Promoter Region: Location and Function of the Upstream Activating Sequence UAS_{G}. Mol Cell Biol November 1984: 2467-2478
Wildt S, and Deuschle U. (1999) cobA, a red fluorescent transcriptional reporter for Escherichia coli, yeast and mammalian cells. Nat Biotech 17: 1175-1178
Wong T. S., Tee K. L., Hauer B., Schwaneberg U. (2004) Sequences saturation mutagenesis (SeSaM): a novel method for directed evolution. Nucleic Acid Research 32(3): e26
Yocum R. R., Hanley S., West R., Ptashne M. (1984) Use of lacZ Fusions to Delimit Regulatory Elements of the Inducible Divergent GAL-1-GAL10 Promoter in Saccharomyces cerevisiae. Mol Cell Biol October 1984: 1985-1998
Zaccolo M., Williams D. M., Brown D. M., Gherardi E. (1996) mAn approach to random mutagenesis of DNA using mixtures of triphosphate derivatives of nucleoside analogues. J. Mol. Biol. 255: 589-603
Zha D., Eipper A., Reetz M. T. (2003) Assembly of Designed Oligonucleotides as an Efficient Method for Gene Recombination: A New Tool in Directed Evolution. chemBioChem 4: 34-39
Zhang J. and Lautar S. (1996) A Yeast Three-Hybrid Method to clone Ternary Protein Complex Components. Anal. Biochem. 242, 68-72
Zheng L., Baumann U., Reymond J-L. (2004) An efficient one-step site-directed and site-saturation mutagenesis protocol. Nucleic Acids Research 32(14): e115

1. A method of identifying high-affinity ligands, comprising the following steps:
a) generating a library for a mutagenized first hybrid protein, comprising a multiplicity of mutants;
b) expressing said first hybrid protein in a host with a second hybrid protein, with one of said hybrid proteins comprising the DNA binding domain of a transcription factor and a bait protein, and the other hybrid protein comprising the activating domain for a transcription factor and a prey protein;
c) enabling said first and second hybrid proteins to bind to one another to give a complex containing a functional transcription factor in the host cell under reaction conditions chosen so as to shift the equilibrium of the binding reaction toward the side of the hybrid proteins;
d) detecting the binding reaction by detecting a reporter gene expressed via the functional transcription factor;
e) optionally repeating one or more steps from a) to d);
f) selecting a mutant.

2. The method as claimed in claim 1 wherein the equilibrium is shifted via the ionic strength of the reaction medium.

3. The method as claimed in claim 1, wherein the equilibrium is shifted via the pH of the reaction medium.

4. The method as claimed in claim 1, wherein the equilibrium is shifted via usage of a competitor of at least one hybrid protein.

5. The method as claimed in claim 4, wherein the concentration of the competitor is varied.

6. The method as claimed in claim 4, wherein the competitor is expressed in the host cell.

7. The method as claimed in claims 4, wherein expression of the competitor is regulated by way of choosing a suitable promoter.

8. The method as claimed in any of claims 1, wherein the host cell is cultured on a selection medium.

9. The method as claimed in claim 1 wherein the binding reactions of at least two different hybrid proteins are compared to one another.

10. The method as claimed in claim 9, wherein the binding reaction of a mutagenized hybrid protein is compared to the binding reaction of its wild type.

11. The method as claimed in claim 9, wherein the binding reaction of a mutagenized hybrid protein is compared to the binding reaction of a mutagenized protein derived from the wild type.

12. A host cell coding for a first hybrid protein and a second hybrid protein, it being possible for said hybrid proteins to form together a functional ligand complex, and a protein which is a competitor of either of said hybrid proteins.

13. A system of a host cell coding for a first hybrid protein and a second hybrid protein, it being possible for said hybrid proteins to form together a functional ligand complex, and of a protein which is a competitor of either of said hybrid proteins.

14. A plasmid coding for a first hybrid protein which, together with a second hybrid protein, forms a ligand complex, and for another protein which is a competitor of said first or second hybrid protein.

15. The use of the host cell as claimed in claim 12 for determining binding affinities.

16. The use of a fluorophore whose maximum emission is between 550 and 700 nm, preferably between 580 and 650, particularly preferably between 600 and 620, especially preferably between 600 and 610 nm as readout in yeasts in which at least two hybrid proteins are coexpressed.

17. The use as claimed in claim 16, wherein the fluorophore is formed by a reporter gene coexpressed in the yeast.

18. The use as claimed in either of claim 16 wherein the fluorophore is phycocyanine or RedStar.

19. The use as claimed in either of claim 16, wherein the fluorophore is a uroporphyrinogene III derivative.

20. The use as claimed in claim 19, wherein the fluorophore is formed by a reporter gene encoded by any of the following genes or genes homologous thereto: CobA, Met1, CysG.

21. The use as claimed in claim 20, wherein the reporter gene has any of the following sequences: SEQ ID NO 341 35, 36 and 37.

22. A fluorophore encoded by any of the following sequences: SEQ ID NO 35, 36 and 37 or sequences homologous thereto.
